(19) United States
(12) Patent Application Publication
(10) Pub. No.: US 2025/0312387 A1
(43) Pub. Date: Oct. 9, 2025

(54) BACTERIAL THERANOSTIC RADIOIMMUNOTHERAPY

(71) Applicants: University of Massachusetts, Boston, MA (US); Cornell University, Ithaca, NY (US)

(72) Inventors: Lars Howell, Boston, MA (US); Neil Forbes, Boston, MA (US); Sarah Taldone, Ithaca, NY (US)

(21) Appl. No.: 19/173,895

(22) Filed: Apr. 9, 2025

Related U.S. Application Data
(60) Provisional application No. 63/631,748, filed on Apr. 9, 2024.

(51) Int. Cl. A61K 35/74; C07K 14/195; C07K 16/44; C12N 1/20; C12N 15/74; C12R 1/42

(52) U.S. Cl. CPC A61K 35/74; C07K 14/195; C07K 16/44; C12N 1/20; C12N 15/74; C07K 2317/622; C07K 2319/01; C12R 2001/42

(57) ABSTRACT

Disclosed herein is an engineered bacterium comprising a chimeric nucleic acid comprising: a first nucleic acid encoding an ompA protein or a fragment thereof; and a second nucleic acid encoding a radiohapten binding protein. Further disclosed are methods of using these constructs in methods for pre-targeted radiotherapy and for treating tumors.

### CROSS REFERENCE TO RELATED APPLICATIONS

This application claims the benefit of U.S. Provisional Patent Application 63/631,748, filed Apr. 9, 2024, the disclosure of which is hereby incorporated by reference in its entirety.

### FIELD OF THE INVENTION

Disclosed herein are chimeric nucleic acid and engineered bacterium compositions and methods of using thereof in methods for pre-targeted radiotherapy and for diagnosing and treating tumors.

### INCORPORATION BY REFERENCE

The contents of the xml file named “11555-009US1-ST26” which was created on Apr. 3, 2025, and is 69.7 KB in size, are hereby incorporated by reference in their entirety.

### BACKGROUND

Radiopharmaceutical therapy (RPT) is a potent first-line therapy for multiple cancer types. In RPT, a therapeutic radionuclide directly delivers ionizing radiation to malignant tissue by specific localization. However, RPT is limited in solid tumors by low therapeutic indices (TIs) causing objective responses in only 30-60% of patients.

As such, there is an urgent need for improved RPT targeting and localization, particularly in solid tumors.

### SUMMARY

It has been surprisingly and unexpectedly discovered that a chimeric nucleic acid expressing an chimeric protein comprising ompA and a radiohapten binding protein and engineered bacteria expressing thereof via surface display effectively bind a radiohapten, for example including but not limited to the radionucleotide DOTA.

Disclosed herein are bacterial theranostic radioimmunotherapy (B-TRIT) engineered bacteria and engineered construct compositions, and methods of using thereof in methods for pre-targeted radiotherapy and for diagnosing and/or treating tumors.

The result is remarkable in that the surface display of chimeric proteins as described herein provides one or more of: (i) enhanced periplasmic localization of the chimeric protein; (ii) enhanced protein folding of the radiohapten binding protein; (iii) increased binding of a radiohapten, for example including but not limited to the radionucleotide DOTA; (iv) increased tumor localization; and/or (v) increased intratumoral DOTA accumulation in vivo.

This improvement provides a significant advance in the state of the art of pre-targeted radioimmunotherapy for the diagnosis and/or treatment of solid tumors.

In contrast, it has been discovered that the profound benefits achieved using the chimeric nucleic acids and chimeric proteins encoded therefrom as described herein are not achieved when a bacterium expresses a radiohapten binding protein alone, for example including but not limited to the anti-DOTA scFV 2D12.5, wherein poor surface display and/or bacterial toxicity is observed.

Specific benefits of the chimeric nucleic acids, chimeric proteins, and plasmids and/or vectors or engineered bacteria expressing thereof as described herein include one or more of:
(i) enhanced surface display and/or periplasmic localization of the radiohapten binding protein, including but not limited to the anti-DOTA scFv 2D12.5 (DBP), via fusion with OmpA (see, e.g., FIGS. 2A, 5);
(ii) increased binding of the chimeric protein to the radiohapten, including but not limited to DOTA (see, e.g., FIGS. 2D-2E), which was dependent upon surface display of the radiohapten binding protein (see, e.g., FIG. 5);
(iii) improved folding and binding ability of the radiohapten binding protein, including but not limited to the anti-DOTA scFv 2D12.5 (DBP), to the radiohapten (e.g., DOTA), via coexpression of a heterologous nucleic acid encoding for a disulfide bond forming protein (dsbA) (see, e.g., FIG. 3A-3C);
(iv) improved binding kinetics of engineered bacteria comprising a chimeric nucleic acid, and/or expressing the chimeric protein, as described herein (see, e.g., FIG. 3C-3D);
(v) enhanced tumor accumulation of the engineered bacteria comprising a chimeric nucleic acid, and/or expressing the chimeric protein, as described herein (see, e.g., FIG. 4A-4B); and/or
(vi) increased DOTA localization to tumors via injection of engineered bacteria comprising a chimeric nucleic acid, and/or expressing the chimeric protein, as described herein (see, e.g., FIG. 4C-4D).

In one aspect, disclosed herein is a chimeric nucleic acid for radiohapten delivery and engineered bacteria expressing the same. In some embodiments, the engineered bacterium, e.g., Salmonella typhimurium, can display on its surface a radiohapten binding protein via fusion to a subset of the ompA protein, which can target and localize radiotherapies (e.g., DOTA) to a tumor environment. In some embodiments, the engineered bacterium can overexpress the protein dsbA to enhance folding of the protein and DOTA binding in vitro.

In an aspect, provided is a chimeric nucleic acid including a first nucleic acid encoding an ompA protein or a fragment thereof; and a second nucleic acid encoding a radiohapten binding protein.

In another aspect, provided is a plasmid or a vector comprising any of the disclosed chimeric nucleic acids.

In another aspect, provided is a cell comprising any of the disclosed chimeric nucleic acids.

In another aspect, provided is a chimeric protein, wherein the chimeric protein is encoded by any of the disclosed chimeric nucleic acids.

In another aspect, provided is an engineered bacterium, comprising any of the disclosed chimeric nucleic acids or other nucleic acids as described herein.

In another aspect, provided is an engineered bacterium, expressing any of the disclosed chimeric proteins or other proteins as described herein.

In another aspect, provided is a method of pre-targeted radiotherapy in a subject, including administering to the subject any of the disclosed engineered bacteria.

In another aspect, provided is a method of treating a tumor in a subject in need thereof, including administering to the subject any of the disclosed engineered bacteria.

In another aspect, provided is a method of diagnosing a tumor and/or a cancer in a subject in need thereof, including administering to the subject any of the disclosed engineered bacteria.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A-1D show multiple surface display methods were tested for bacterial expression of the scFv 2D12.5 and DOTA Binding.

FIG. 1A shows the scFv 2D12.5 (DBP) and a flag tag was fused to the outermost protein of Salmonella fimbrae, fimH to form Sal-f-DBP. DBP was fused to the passenger domain of the Salmonella autotransporter protein misL to form Sal-m-DBP. DBP was fused to the five 5 β-sheets of the Salmonella OmpA protein to form Sal-o-DBP. Surface expression of DOTA binding protein (DBP) performed by a fusion of 5 β-sheets of the Salmonella ompA protein with a flag tag for detection and the 2D12.5 scFv fragment, all pieces are separated by 3 G4S1 flexible protein linkers.

FIG. 1B shows structures of the gene fusions for the constructs. The flag tag was separated from the 2D12.5 DBP and from the surface display protein with three G4S1 flexible linker sequences.

FIG. 1C shows a plasmid construct comprising nucleic acids encoding for DBP, asd, and dsbA, driven by arabinose inducible promoter (pBAD), pASD, and pBAD promoters, respectively.

FIG. 1D shows binding of DOTA was analyzed via flow cytometry after allowing the Salmonella to bind in a solution of 100 nM DOTA for 3 hours (n=3). Sal-o-DBP binds to DOTA in vitro more than Sal-f-DBP (****, P<0.0001) or Sal-m-DBP (****, P<0.0001). Sal-f-DBP bound more effectively than Sal-m-DBP which had negligible binding (*, P=0.305). Data are represented as mean+/−SEM.

FIGS. 2A-2F show that Sal-o-DBP localizes OmpA-2D12.5 to the periplasm and binds to DOTA in vitro.

FIG. 2A shows expression of the 47 kDa protein validated by western blotting. Localization of the DBP protein within the bacteria overtime is performed by comparing the protein in the periplasmic isolation of the bacteria and the whole cell fraction at 1, 2, 4, and 6 hours post induction. Samples were analyzed via western blotting staining for flag tag expression.

FIGS. 2B-2C show quantification of the western bands in FIG. 2A and fractional calculation of the periplasmic portion.

FIGS. 2D-E show that, when cultured in a 100 nM DOTA solution, induced Sal-o-DBP binds to DOTA with 30 percent of cells bound, while uninduced Sal-o-DBP has negligible binding (****, P<0.0001).

FIG. 2F shows that, to determine binding specificity ratios of uninduced to induced, Sal-o-DBP were bound to 100 nM DOTA before being analyzed via flow cytometry (n=3). Each ratio of uninduced to induced Salmonella bound more than the lower ratio (**, P<0.01) except 1:1 to 1:3. There was a linear relationship between the fraction of induced Sal-o-DBP and the fluorescent intensity (m=1.007, R²=0.9568). Data are represented as mean+/−SEM.

FIGS. 3A-3D show that adding disulfide bond forming protein (dsbA) increases DOTA binding.

FIG. 3A shows the addition of dsbA increases Sal-o-DBP binding to DOTA after mixing with 100 nM DOTA (n=3; P=*, 0.0103).

FIG. 3B shows example histograms demonstrating DOTA binding fluorescent intensity with and without dsbA in i) 100 nM DOTA or ii) 10 pM DOTA. Left of the black line is unbound Salmonella, right indicates DOTA bound Salmonella.

FIG. 3C shows binding affinity of Sal-DBP at 37° C. of Sal-o-DBP with (square points) and without dsbA (circle points) via a direct binding assay (n=3). Adding dsbA Sal-o-DBP increases DOTA binding at 100 nM (****, P<0.0001), 10 nM (****, P<0.0001), 1 nM (****, P<0.0001), and 100 pM (*, P=0.0415).

FIG. 3D shows a dissociation curve of Sal-DBP with dsbA which was measured by direct binding after various dissociation times at 37° C. (n=3). Data are represented as mean+/−SEM, n=3.

FIGS. 4A-4D show that Sal-o-DBP localizes to and accumulates DOTA in tumors.

FIG. 4A shows biodistribution of bacteria in tumor, liver, spleen, and kidneys 54 hours after intratumoral injection (n=4) of Sal-o-DBP. Salmonella accumulate more in tumors than in livers, spleens, or kidneys (**, P=0.010).

FIG. 4B shows example immunohistochemical staining of tumors shows that injection of Sal-o-DBP (left) results in Salmonella distribution throughout the tumor when compared to tumors without bacterial injection (right).

FIG. 4C shows the concentration of DOTA in the tumors after injection with saline or Sal-o-DBP. Injection of Sal-o-DBP increases DOTA entrapment in the tumor compared to mice without bacterial injections (*, P=0.0468). The top point in Sal-o-DBP is a statistical outlier and was not used when calculating the P value.

FIG. 4D shows the biodistribution of DOTA two hours after intravenous injection of 10 nmol DOTA in mice injected intratumorally with Sal-o-DBP. Data are represented as mean+/−SEM.

FIG. 5 shows a model of engineered Salmonella for pre-targeted radioimmunotherapy to solid tumors.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

As used herein, the term “about” means±10%.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention, “substantially complementary” refers to about 90% or greater identity over the length of the target base pair region. The complementarity can also be about 50, about 60, about 70, about 75, about 80, about 85, about 90, about 91, about 92, about 93, about 94, about 95, about 96, about 97, about 98, about 99, or 100% complementary, or any amount below or in between these amounts.

As used herein, “nucleic acid sequence” refers to the order or sequence of nucleotides along a strand of nucleic acids. In some cases, the order of these nucleotides may determine the order of the amino acids along a corresponding polypeptide chain. The nucleic acid sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc. It may include modified bases, including locked nucleic acids, peptide nucleic acids and others known to those skilled in the art.

An “oligonucleotide” is a polymer comprising two or more nucleotides. The polymer can additionally comprise non-nucleotide elements such as labels, quenchers, blocking groups, or the like. The nucleotides of the oligonucleotide can be natural or non-natural and can be unsubstituted, unmodified, substituted or modified. The nucleotides can be linked by phosphodiester bonds, or by phosphorothioate linkages, methylphosphonate linkages, boranophosphate linkages, or the like.

A “primer” is a nucleic acid that contains a sequence complementary to a region of a template nucleic acid strand and that primes the synthesis of a strand complementary to the template (or a portion thereof). Primers are typically 18-20 base long, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified.

The “Tm” (melting temperature) of a nucleic acid duplex under specified conditions is the temperature at which half of the nucleic acid sequences are disassociated and half are associated. As used herein, “isolated Tm” refers to the individual melting temperature of either the first or second nucleic acid sequence in the cooperative nucleic acid when not in the cooperative pair. “Effective Tm” refers to the resulting melting temperature of either the first or second nucleic acid when linked together.

As used herein, “amplify, amplifying, amplifies, amplified, amplification” refers to the creation of one or more identical or complementary copies of the target DNA. The copies may be single stranded or double stranded. Amplification can be part of a number of processes such as the extension of a primer, reverse transcription, polymerase chain reaction, nucleic acid sequencing, rolling circle amplification and the like.

As used herein, “purified” refers to a polynucleotide, for example a target nucleic acid sequence, that has been separated from cellular debris, for example, high molecular weight DNA, RNA and protein. This would include an isolated RNA sample that would be separated from cellular debris, including DNA. It can also mean non-native, or non-naturally occurring nucleic acid.

As used herein, “protein,” “peptide,” and “polypeptide” are used interchangeably to denote an amino acid polymer or a set of two or more interacting or bound amino acid polymers.

As used herein, “stringency” refers to the conditions, i.e., temperature, ionic strength, solvents, and the like, under which hybridization between polynucleotides occurs. Hybridization is the process that occurs between the primer and template DNA during the annealing step of the amplification process.

As used herein, “multiplex” refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

The term “sensitivity” refers to a measure of the proportion of actual positives which are correctly identified as such.

The term “confidence level” refers to the likelihood, expressed as a percentage, that the results of a test are real and repeatable and not random. Confidence levels are used to indicate the reliability of an estimate and can be calculated by a variety of methods.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain. In certain embodiments, sequences of the present invention, including primer sequences, target sequences and internal amplification control (IAC) sequences may be identical to the sequences provided here in or comprise less than 100% sequence identity to the sequences provided herein. For instance, primer sequences, target sequences or IAC sequences of the present invention may comprise 90-100% identity to the sequences provided herein.

The terms “identical” or “percent identity,” in the context of two or more nucleic acids or sequences, refer to two or more sequences or subsequences that are the same or have a specified percentage of nucleotides that are the same (i.e., at least about 60% identity, preferably at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated regions that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has” or “includes” one or more steps is not limited to possessing only those one or more steps and also covers other unlisted steps. Similarly, any cell that “comprises,” “has” or “includes” one or more traits is not limited to possessing only those one or more traits and covers other unlisted traits.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures that can perform the same function which are related to the disclosed structures and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### Compositions and Methods

In an aspect, provided is a chimeric nucleic acid including (a) a first nucleic acid encoding an ompA protein or a fragment thereof; and (b) a second nucleic acid encoding a radiohapten binding protein.

In some aspects, the radiohapten binding protein comprises a 2,2′,2″,2″′-(1,4,7,10-tetraazacyclododecane-1,4,7,10-tetrayl)tetraacetic acid (DOTA)-binding protein.

In some aspects, the DOTA-binding protein comprises an anti-DOTA single chain variable fragment (scFV).

In some aspects, the anti-DOTA scFV comprises the amino acid sequence of SEQ ID NO: 1, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 1.

In some aspects, the anti-DOTA scFV is encoded by SEQ ID NO: 2, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 2.

In some aspects, the DOTA-binding protein comprises an anti-DOTA single chain variable fragment (scFV), when expressed in combination with dsbA, comprises a first disulfide bond between Cys220 and Cys293.

In some aspects, the DOTA-binding protein comprises an anti-DOTA single chain variable fragment (scFV), when expressed in combination with dsbA, comprises a second disulfide bond between Cys354 and Cys422.

In some aspects, the DOTA-binding protein comprises an anti-DOTA single chain variable fragment (scFV), when expressed in combination with dsbA, comprises a first disulfide bond between Cys220 and Cys293 and a second disulfide bond between Cys354 and Cys422.

In some aspects, the radiohapten binding protein binds to a radioactive isotope selected from actinium-225, astatine-211, bismuth-212, bismuth-213, carbon-14, cerium-134, chromium-51, chlorine-36, cobalt-57, cobalt-58, copper-64, copper-67, europium-152, fluorine-18, gallium-67, gallium-68, hydrogen-3, iodine-123, iodine-124, iodine-125, iodine-131, indium-111, iron-59, lanthanum-132, lanthanum-135, lead-203, lead-212, lutetium-177, phosphorus-32, radium-223, radium-224, rhenium-186, rhenium-188, scandium-43, scandium-44, scandium-47, selenium-75, sulphur-35, technicium-99m, terbium-149, terbium-152, terbium-155, terbium-161, thorium-227, yttrium-86, yttrium-90, zirconium-89, or any combination thereof.

In some aspects, the ompA protein or a fragment thereof is operably linked to the N-terminus of the radiohapten binding protein.

In some aspects, the ompA protein or a fragment thereof is operably linked to the C-terminus of the radiohapten binding protein.

In some aspects, the chimeric nucleic acid further comprises an additional nucleic acid sequence encoding for one or more linkers.

In some aspects, the ompA protein or a fragment thereof is operably linked to the radiohapten binding protein by one or more linkers. In some aspects, the ompA protein or a fragment thereof is operably linked to the radiohapten binding protein by one or more G4S1 linkers.

In some aspects, the ompA protein or a fragment thereof comprises the amino acid sequence of SEQ ID NO: 3, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 3.

In some aspects, the ompA protein or a fragment thereof is encoded by SEQ ID NO: 4, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 4.

In some aspects, the linker comprises the amino acid sequence of SEQ ID NO: 5 (GGGGSGGGGSGGGGS), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 5.

In some aspects, the linker is encoded by SEQ ID NO: 6, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 6.

In some aspects, the linker comprises the amino acid sequence of SEQ ID NO: 7 (GGGGSGGGGRGGGGR), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 7.

In some aspects, the linker is encoded by SEQ ID NO: 8, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 8.

In some aspects, the linker further comprises an epitope tag for protein detection. In some aspects, the epitope tag comprises the amino acid sequence of SEQ ID NO: 9, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 9.

In some aspects, the epitope tag is encoded by SEQ ID NO: 10, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 10.

In some aspects, the linker comprises the amino acid sequence of SEQ ID NO: 11, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 11.

In some aspects, the linker is encoded by SEQ ID NO: 12, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 12.

In some aspects, the linker comprises any combination of glycine and serine amino acids. In some such aspects, the linker can be from 1 amino acid to 30 amino acids (e.g., from 1 amino acid to 29 amino acids, from 2 amino acids to 28 amino acids, from 3 amino acids to 27 amino acids, from 4 amino acids to 26 amino acids, from 5 amino acids to 25 amino acids, from 6 amino acids to 24 amino acids, from 7 amino acids to 23 amino acids, from 8 amino acids to 22 amino acids, from 9 amino acids to 21 amino acids, from 10 amino acids to 20 amino acids, from 11 amino acids to 19 amino acids, from 12 amino acids to 18 amino acids, from 13 amino acids to 17 amino acids, from 14 amino acids to 16 amino acids, from 1 amino acid to 15 amino acids, from 2 amino acids to 14 amino acids, from 3 amino acids to 13 amino acids, from 4 amino acids to 12 amino acids, from 5 amino acids to 11 amino acids, from 6 amino acids to 10 amino acids, from 7 amino acids to 9 amino acids, from 15 amino acids to 30 amino acids, from 16 amino acids to 29 amino acids, from 17 amino acids to 28 amino acids, from 18 amino acids to 27 amino acids, from 19 amino acids to 26 amino acids, from 20 amino acids to 25 amino acids, from 21 amino acids to 24 amino acids, from 22 amino acids to 23 amino acids).

In some aspects, the linker has a ratio of glycine to serine of about 1:1, about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:2, or about 2:1.

In some aspects, the chimeric nucleic acid comprises the sequence SEQ ID NO: 13 (ompA-DBP sequence), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 13.

In some aspects, the chimeric nucleic acid encodes for the amino acid sequence of SEQ ID NO: 14 (ompA-DBP sequence), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 14.

In some aspects, the chimeric nucleic acid further comprises a first heterologous nucleic acid encoding a dsbA protein.

In some aspects, dsbA is encoded by SEQ ID NO: 15, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 15.

In some aspects, dsbA comprises the amino acid sequence of SEQ ID NO: 16, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 16.

In some aspects, the chimeric nucleic acid further comprises a second heterologous nucleic acid comprising the plasmid retention gene asd.

In some aspects, asd is encoded by SEQ ID NO: 17, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 17.

In some aspects, the chimeric nucleic acid further comprises a second heterologous nucleic acid encoding an lpp motif.

In some aspects, the lpp motif comprises the amino acid sequence of SEQ ID NO: 26, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 26.

In some aspects, the lpp motif is encoded by SEQ ID NO: 27, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 27.

In another aspect, provided is a plasmid or vector comprising any of the disclosed chimeric nucleic acids. In some embodiments, the first and/or second nucleic acids are under the control of one or more promoters. In some embodiments, the one or more promoters are selected from pBAD, pASD, or a combination thereof.

The term “promoter”, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of the same.

In embodiments, the promoter is selected from a T7 promoter, an Sp6 promoter, a lac promoter, an araBad promoter, a trp promoter, or a Ptac promoter.

In embodiments, the promoter is an inducible promoter. In embodiments, the inducible promoter is selected from a pLac promoter, pTac promoter, a tetracycline-controlled promoter, or a pBAD promoter. In embodiments, the inducible promoter is a pLac promoter. In embodiments, the inducible promoter is a pTac promoter. In embodiments, the inducible promoter is a tetracycline-controlled promoter. In embodiments, the inducible promoter is a pBAD promoter.

In embodiments, the promoter is a tumor-specific promoter.

In another aspect, provided is a cell including any of the disclosed chimeric nucleic acids.

In another aspect, provided is a cell comprising any of the disclosed plasmids or vectors.

In some aspects, the plasmid comprises the nucleic acid sequence of SEQ ID NO: 28, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 28.

In some aspects, the plasmid comprises the nucleic acid sequence of SEQ ID NO: 29, or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 29.

In another aspect, provided is a chimeric protein, wherein the chimeric protein is encoded by any of the disclosed chimeric nucleic acids.

In some aspects, the chimeric protein comprises the amino acid sequence SEQ ID NO: 14 (ompA-DBP sequence), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 14.

In another aspect, provided is an engineered bacterium, including any of the disclosed chimeric nucleic acids.

In another aspect, provided is an engineered bacterium, expressing any of the disclosed chimeric proteins.

In some aspects, the DBP, when co-expressed with ompA, is presented on the outer surface of the engineered bacterium. Advantageously, surface expressed DBP preferentially binds DOTA.

In some aspects, the DBP, when co-expressed with ompA, is expressed in the periplasm of the engineered bacterium.

In some aspects, the DBP, when co-expressed with dsbA, is modified by one or more disulfide bonds in the oxidizing environment of periplasm of the engineered bacterium.

In some aspects, the engineered bacterium is selected from a Salmonella bacterium, a Listeria bacterium, an Escherichia bacterium, a Clostridium bacterium, a Yersinia bacterium, a Mycobacterium, a Streptococcus bacterium, a Bifidobacterium, a Lactobacillus bacterium, or a Limosilactobacillus bacterium. In some aspects, the engineered bacterium comprises a Salmonella typhimurium bacterium (e.g., Salmonella enterica serovar Typhimurium). In some aspects, the engineered bacterium comprises an Escherichia coli (E. coli) bacterium. In some aspects, the engineered bacterium comprises a Listeria monocytogenes (L. monocytogenes) bacterium. In some aspects, the engineered bacterium comprises a Bifidobacterium infantis bacterium. In some aspects, the engineered bacterium comprises a Bifidobacterium longum bacterium. In some aspects, the engineered bacterium comprises a Lactobacillus acidophilus bacterium. In some aspects, the engineered bacterium comprises a Lactobacillus rhamnosus bacterium. In some aspects, the engineered bacterium comprises a Limosilactobacillus fermentum bacterium.

In some aspects, the Salmonella bacterium comprises Salmonella VNP20009.

In some aspects, the engineered bacterium comprises the sequence SEQ ID NO: 13 (ompA-DBP sequence), or a sequence at least about 70% identical (e.g., at least about 71% identical, at least about 72% identical, at least about 73% identical, at least about 74% identical, at least about 75% identical, at least about 76% identical, at least about 77% identical, at least about 78% identical, at least about 79% identical, at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical, or more) to SEQ ID NO: 13.

In some aspects, the engineered bacterium further comprises a heterologous nucleic acid encoding a dsbA protein.

In some aspects, provided herein is a pharmaceutical composition comprising at least engineered bacterium, and a pharmaceutically acceptable carrier and/or excipient.

As used herein a “pharmaceutical composition” refers to a preparation of engineered bacteria of the disclosure with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered engineered bacteria. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

In some embodiments, the nucleotide sequences disclosed herein are codon optimized nucleotide sequences.

In another aspect, provided is a method of pre-targeted radiotherapy in a subject, including administering to the subject any of the disclosed engineered bacteria.

In another aspect, provided is a method of treating a tumor in a subject in need thereof, the method comprising administering to the subject any of the disclosed engineered bacteria.

Any appropriate route or mode of administration to the subject can be employed according to a method provided herein. In some cases, administering comprises oral administration of the engineered bacterium. In other cases, administering comprises intra-tumoral injection of the engineered bacterium. The mode of administration can be determined based on the physical location, type, or number of tumors in the subject's body.

Clinicians, physicians, and other health care professionals can administer engineered bacteria to a subject in need thereof according to a method provided herein. In some cases, a single administration of the composition may be sufficient. In other cases, more than one administration of the composition is performed at various intervals (e.g., once per week, twice per week, daily, monthly) or according to any other appropriate treatment regimen. The duration of treatment can be a single dose or periodic multiple doses for as long as the administration of a composition provided herein is tolerated by the subject.

In some aspects, the engineered bacteria are administered by intravenous injection.

In some aspects, the engineered bacteria are administered by intratumoral injection.

In some aspects, the engineered bacteria preferentially inhabit a tumor microenvironment (TME) following administration and/or injection.

In another aspect, provided is a method of diagnosing a tumor and/or a cancer in a subject in need thereof, the method comprising administering to the subject any of the disclosed engineered bacteria.

In another aspect, provided is a method of diagnosing and treating a tumor in a subject in need thereof, the method comprising administering to the subject any of the disclosed engineered bacteria.

In some aspects, the tumor is a solid tumor. In some aspects, the tumor comprises a sarcoma or a carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma).

### Embodiments

1. A chimeric nucleic acid comprising:
a first nucleic acid encoding an ompA protein or a fragment thereof; and
a second nucleic acid encoding a radiohapten binding protein.
   2. The chimeric nucleic acid of embodiment 1, wherein the radiohapten binding protein is a DOTA (2,2′,2″,2″′-(1,4,7,10-tetraazacyclododecane-1,4,7,10-tetrayl)tetraacetic acid)-binding protein.
   3. The chimeric nucleic acid of embodiment 2, wherein the DOTA-binding protein is an anti-DOTA scFV.
   4. The chimeric nucleic acid of any one of embodiments 1 to 3, wherein the anti-DOTA scFV is encoded by SEQ ID NO: 2, or a sequence at least 70% identical to SEQ ID NO: 2.
   5. The chimeric nucleic acid of any one of embodiments 1 to 4, wherein the ompA protein or a fragment thereof is operably linked to the N-terminus of the radiohapten binding protein.
   6. The chimeric nucleic acid of any one of embodiments 1 to 5, wherein the ompA protein or a fragment thereof is operably linked to the radiohapten binding protein by a linker.
   7. The chimeric nucleic acid of any one of embodiments 1 to 6, wherein the ompA protein or a fragment thereof is encoded by SEQ ID NO: 4, or a sequence at least 70% identical to SEQ ID NO: 4.
   8. The chimeric nucleic acid of any one of embodiments 1 to 7, wherein the linker comprises SEQ ID NO: 5 (GGGGSGGGGSGGGGS).
   9. The chimeric nucleic acid of any one of embodiments 1 to 8, wherein the nucleic acid comprises the sequence SEQ ID NO: 13 (ompA-DBP sequence), or a sequence at least 70% identical to SEQ ID NO: 13.
   10. A cell comprising the chimeric nucleic acid of any one of embodiments 1 to 9.
   11. A chimeric protein, wherein the chimeric protein is encoded by the chimeric nucleic acid of any one of embodiments 1 to 9.
   12. An engineered bacterium, comprising the chimeric nucleic acid of any one of embodiments 1 to 9.
   13. The engineered bacterium of embodiment 12, wherein the engineered bacterium is a Salmonella bacterium.
   14. The engineered bacterium of embodiment 13, wherein the engineered bacterium is a Salmonella typhimurium bacterium.
   15. The engineered bacterium of any one of embodiments 12 to 14, wherein the engineered bacterium comprises the sequence SEQ ID NO: 13 (ompA-DBP sequence), or a sequence at least 70% identical to SEQ ID NO: 13.
   16. The engineered bacterium of any one of embodiments 12 to 15, further comprising a heterologous nucleic acid encoding a dsbA protein.
   17. A method of pre-targeted radiotherapy in a subject, comprising administering to the subject the engineered bacterium of any one of embodiments 12 to 16.
   18. A method of treating a tumor in a subject in need thereof, comprising administering to the subject the engineered bacterium of any one of embodiments 12 to 16.
   19. The method of embodiment 18, wherein the tumor is a solid tumor.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Engineered Salmonella for Pre-Targeted Radioimmunotherapy to Solid Tumors

Engineered bacteria have a high tumor-to-normal tissue ratio and overcoming the limitations in RPT. Disclosed herein is a system of Bacterial Theranostic Radioimmunotherapy (B-TRIT). This technique is similar to pre-targeted radioimmunotherapies (PRIT), a multi-step RPT technique that utilizes a non-radioactive targeting vector to localize to the tumor, followed by a radioactive payload with rapid clearance. The targeting vector must localize to the tumor and be bind a radiolabeled small molecule to capture it. However, many PRIT systems utilize bispecific antibodies to perform this double binding, targeting both a tumor antigen and the radiohapten. The limitation of this system is that the bispecific antibody must be highly specific, clear rapidly from blood and non-tumorous tissue, and perfuse into the highly vascularized tumor tissue. Antigen expression is highly variable among tumor types and is patient specific, also limiting PRIT. Engineered bacteria can serve as an antigen-independent tumor targeting system as they have a high tumor-to-normal tissue ratio and target a wide variety of tumor types. Salmonella exit the body through hepatic clearance10 while the renal system is the most sensitive to radiation damage. Utilizing two systems with orthogonal clearance mechanisms for delivery would increase the therapeutic index. The advantage of the B-TRIT systems is that it is tumor agnostic and does not rely on knowledge of tumor antigen presentation which is variable and patient specific. Here, we describe a system of Bacterial PRIT (B-PRIT) that utilizes bacterial display of the radiohapten capture antigen.

DOTA ((2,2′,2″,2″′-(1,4,7,10-tetraazacyclododecane-1,4,7,10-tetrayl)tetraacetic acid)) is an ideal radiohapten for PRIT systems due to its safe clinical profile and prior clinical use. DOTA-binding protein (DBP) 2D12.5 is a monoclonal anti-DOTA(radiometal) antibody that binds to DOTA chelates of yttrium and lutetium with low nanomolar affinity. DOTA chelates to a wide variety of metals including ⁹⁰Yttrium, ¹⁷⁷Lutecium, and ²²⁵Actinium allowing it to be used for both diagnostic and therapeutic purposes. DOTA(radiometal) chelates are used as haptens in pre-targeting applications because they possess favorable pharmacokinetic and clinical safety profiles. After intravenous (IV) infusion into mice, DOTA-based haptens have low tissue and whole-body retention, with 2-4% of the injected dose remaining 4 h after injection. An anti-DOTA scFv has been engineered to be expressed by human embryonic kidney cells as a test platform for characterizing radiohapten capture demonstrating the ability to present the scFv on the cell surface and maintain DOTA binding.

Bispecific antibody systems require knowledge of highly expressed tumor antigens within the tumor environment in order for effective targeting; however, not all tumors have homogeneous neoantigen expression. Bacterial therapies can overcome these limitations and form a tumor-independent PRIT system, bacterial-PRIT (B-PRIT). Salmonella chemotax towards tumors and grow in tumors at a rate of 10000:1 compared to healthy tissue. Bacterial systems have been used to manufacture scFvs, a fusion of the heavy and light chain region of antibodies that form the binding pocket without the greater antibody structure; however, these scFvs remain inside the cell and are not available without destroying the bacterium.

Bacteria are engineered to present molecules on their surface using the lpp-ompA display system. This display system uses five β-sheet membrane crossing domains of the E. coli gene ompA as well as the localization sequence lpp to deliver the fusion protein to the bacterial outer membrane and to anchor the protein to the outer membrane, presenting the fused cargo to the outside of the cell.

In order to effectively bind, 2D12.5 has two disulfide bonds to maintain protein tertiary structure. Enhancing disulfide bond formation is key for enhancing bacterial display of functional scFvs for PRIT. Bacteria have a highly reducing cytoplasmic environment which limits the ability for these disulfide bonds to form; whereas the periplasm is a more oxidizing environment allowing for the formation of disulfide bonds. Within the periplasm, disulfide bond forming protein A (dsbA) introduces disulfide bonds between the cysteine amino acids in the protein structures. In DBP 2D12.5 there are predicted disulfide bonds between Cys220 and Cys293 as well as Cys354 and Cys422 (SCRATCH protein predictor (UCI)).

A study was conducted which developed Salmonella that bind the radionucleotide DOTA to the tumor microenvironment by surface display of DOTA binding protein (Sal-DBP). In this study, three techniques for surface display of DBP were engineered before proceeding with a fusion of DBP to OmpA. Surface display of DBP was engineered via fusion to cell wall crossing 3-sheet domains of the native Salmonella protein ompA (Sal-DBP). The study enhanced disulfide bond formation by enhancing co-production of the protein dsbA to increase protein folding in the periplasmic space of the bacteria. The study determined the binding kinetics of DOTA to Sal-DBP in vitro and demonstrated the tumor localization abilities of Sal-DBP in vivo. This system is highly promising for use in pre-targeted radioimmunotherapies and theranostics as Salmonella do not rely on tumor specific antigens for tumor localization, and bacterial therapies and radiopharmaceutical therapies act and clear through orthogonal pathways improving the therapeutic index.

### Materials and Methods

### Bacterial Strains and Plasmids

Salmonella expression DOTA binding protein (Sal-DBP) was constructed using standard cloning techniques and placed under the arabinose inducible promoter, pBAD. The DOTA binding surface display system (ompA-2D12.5) was designed using the available protein sequence for 2D12.5 (SEQ ID NO: 1) which was reverse-translated to the codon optimized nucleotide sequence for Salmonella using EMBOSS backtranseq and the canonical Salmonella ompA sequence (SEQ ID NO: 4). The fusion protein was synthesized (Twist Bioscience, San Francisco, CA). To make the 2D12.5-misL and fimH-2D12.5 fusions part plasmids were assembled that contained 1) D12.5 2) misL and 3) fimH using Gibson assembly primers (Table 2). The fimH and misL genes were amplified from the Salmonella genome. The part plasmids were then assembled using Golden Gate assembly into a backbone plasmid. The genes dsbA (SEQ ID NO: 15) and asd (SEQ ID NO: 17) were cloned into the plasmid via Gibson assembly (table S1). All cloning was performed in chemically competent dh5a E. coli (New England Biolabs, Ipswich, MA). Plasmid sequences were validated using SNPsaurus LLC. Plasmids were transformed into Salmonella VNP20009 (V9; ΔmsbB, ΔpurI, and Δxyl), or V9 Δasd via electroporation. Transformed bacteria were selected by growing on plates or in media containing 100 μg/mL carbenicillin.

### Protein Production and Localization

To assay DBP expression and localization, Sal-DBP was induced and western blots for the flag epitope were performed. To prepare for immunoblotting, bacteria were induced with 10 mM arabinose for 3 hours and grown to OD600 of 0.8. To isolate the periplasmic fraction 1×10⁸ CFU of bacteria were re-suspended in 200 μL of isolation buffer (200 mM Tris pH7.5 with 10 mM EDTA) for 1 hour and vortexed every 10 minutes. Then the bacteria were spun down at 15,800×g for 10 minutes. The soluble fraction contained the bacterial periplasm while the cytoplasm was contained in the pellet. To analyze, the soluble fraction was added to Laemmli SDS sample buffer (ThermoFisher). For whole cell fractions, equal quantities of bacteria (1×10⁸ CFU) were centrifuged (6000×g), re-suspended in Laemmli SDS sample buffer (ThermoFisher). All samples were boiled at 95° C., separated by SDS PAGE electrophoresis, and transferred onto a PVDF membrane. Membranes were blocked and stained in 5% milk powder with 1% Tween in tris-buffered saline (TBS). The membranes were probed for the flag tag using anti-flag primary antibody (1:1000 dilution) and HRP-conjugated anti-rabbit IgG secondary antibody (1:1000 dilution, R&D systems). Blots were imaged using a chemiluminescent HRP substrate (Millipore). Blots were processed in FIJI/ImageJ. Brightness was uniformly enhanced, and the chemiluminescent blot was overlayed on the ladder image captured manually using upper white light. Relative protein concentration was calculated in ImageJ by measuring the intensity of the respective protein bands on the immunoblot.

### Flow Cytometry

Bacterial bound to DOTA was detected via flow cytometry using (1:500) Alexa-fluor 647 conjugated streptavidin (Biolegend; 405237) in 100 μL extracellular staining buffer (ESB; PBS with 0.1% BSA and 1 mM EDTA) stained for 20 minutes on ice. Cells were then fixed in 4 percent paraformaldehyde plus 1 mM EDTA for 15 minutes before being resuspended in ESB. Stained cells were analyzed on a Novocyte 3000 flow cytometer (ACES Biosciences Inc.) and data was analyzed using Novoexpress 1.4.1 (Aligent Technologies Inc.).

### Kinetic Characterization

### Binding Specificity

DOTA binding specificity to surface displayed 2D12.5 was determined by culturing differing ratios of uninduced and induced bacteria and incubating them with 100 nM DOTA for 3 hours before analyzing via flow cytometry. Binding specificity was calculated by background subtracting and comparing the mean fluorescent indexes (MFI) of the various culture ratios.

### Association Constant, K_{D}

Equilibrium dissociation constants (K_{D}) for bacteria surface expressing scFv were determined. Briefly, Salmonella expressing scFv on their surface were grown, then 1×10⁶ bacterial cells were incubated with varying concentrations of DOTA in triplicate for 3 hours at 37 C to reach equilibrium. Bound DOTA-biotin-Lu was detected via flow cytometry.

### Dissociation Constant, K_{off}

To determine K_{off} 1×10⁶ Salmonella were bound to 100 nM DOTA for 3 hours at 37 C. Cells were then washed and resuspended in PBS with 0.1% BSA and 1 mM EDTA for varying times, in triplicate, at 37 C. Cells were placed on ice until all samples were obtained and were then stained and analyzed via flow cytometry.

### Bacterial Biodistribution

To determine the biodistribution of Sal-o-DBP, three groups of six-week old C57BL/6 mice were injected subcutaneously with 1×10⁵ MC38 tumor cells. When tumors reached approximately 200 mm³, the mice were injected intravenously or intratumorally with 1×10⁷ CFU of Sal-o-DBP in 50 μL of saline, or intravenously with saline. Sixty hours after bacterial injection, mice were injected intraperitoneally (IP) with 100 mg arabinose in 400 μL saline. Sixty-six hours after bacterial injection mice were injected intravenously with 10 nmol DOTA in 50 μL of PBS. Two hours after DOTA injection, mouse tumors, spleens, livers, and kidneys were collected for biodistribution analysis. The organs were weighed before being homogenized in equal volumes of sterile PBS. Serial dilutions of the organ slurry were prepared (tumor 10⁻⁵, spleen, liver, and kidney 10-2) and 50 μL of the dilutions were plated on LB agar plates containing 100 μg/mL carbenicillin and incubated overnight at 37° C. Colonies were counted and the total CFU/g was determined by the formula (colony count)×(dilution factor)×(plated volume).

### Dota Biodistribution and Mass Spectroscopy

To determine the biodistribution of DOTA, the organs were analyzed via inductively coupled plasma mass spectrometry (ICP-MS). Fifty μL of Iridium standard was added to each sample. The organ and water mixture was mixed with one mL of 3:1 nitric acid: hydrogen peroxide overnight. Then 500 μL of aqua regia (3:1 hydrochloric acid: nitric acid) was added. This was repeated twice. The solution was then diluted to 10 mL and run on Perkin-Elmer NexION 350D ICP-MS by the UMass Core Facilities.

### Immunohistochemistry

To analyze biodistribution within the tumor a similar injection protocol was performed and the organs were fixed in paraformaldehyde for forty-eight hours before being stored in ethanol prior to paraffin embedding and sectioning. Tumor sections were stained for Salmonella using standard immunohistochemistry protocols. Briefly, samples were deparaffinized and rehydrated using xylene and ethanol before performing antigen retrieval using sodium citrate buffer for 10 minutes. Samples were then blocked in Tris buffered saline (TBS) with 0.5% BSA. Samples were then stained with Salmonella-HRP antibody (Abcam; 1:200) and incubated overnight before reacting with DAB chromogen. The samples were then counterstained with hematoxylin and fixing under glass coverslips. Slides were imaged at 5× magnification via a Zeiss Axio Observer Z.1 microscope under brightfield imaging. Images were processed for brightness and contrast in ImageJ.

### Statistics and Data Analysis

Statistical significance was determined using Student's t test or one-way ANOVA followed by students t test with Tukey's post-hoc comparison test. For K_{D} growth curves, differences in binding at various concentrations between constructs was analyzed using two-way ANOVA with Sidak multiple comparison test. Lines of best fit and their slopes were performed using simple linear regression. Binding curves and equilibrium binding parameters (K_{D} and Bₘₐₓ) were plotted and computed using least-squares non-linear regression via a one-site specific binding model. Bₘₐₓ is the mean fluorescent intensity (MFI) of the Salmonella measured by APC-streptavidin bound to biotin-DOTA. Dissociation curves and parameters (K_{off}) were plotted and computed using least squares regression via Dissociation—One phase exponential decay model. All analysis was performed using GraphPad Prism (GraphPad Software).

### Results

Fusion with OmpA is the Optimal Surface Display Method for the scFv 2D12.5

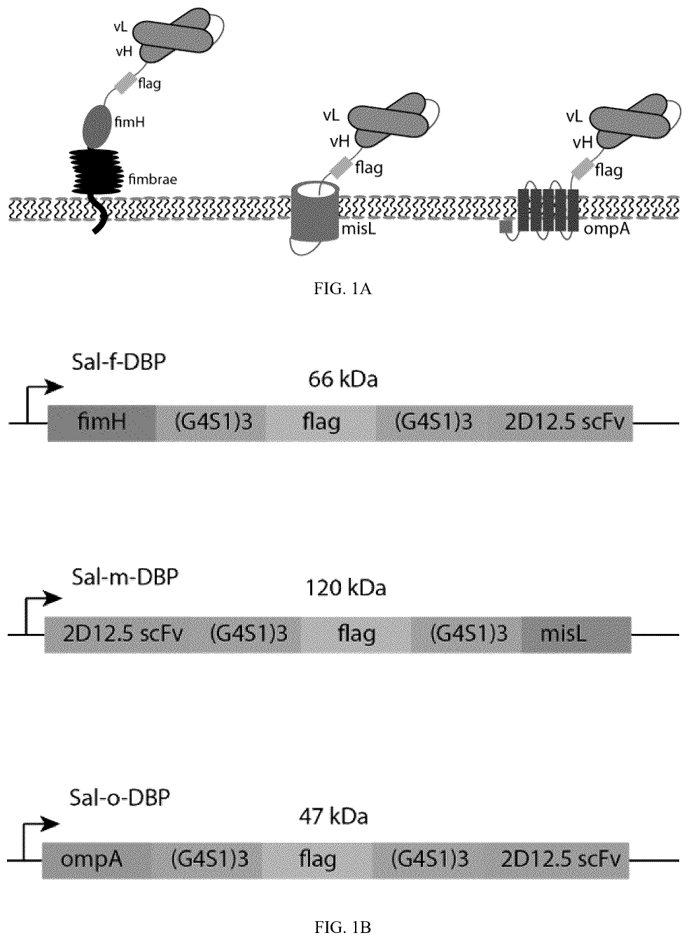

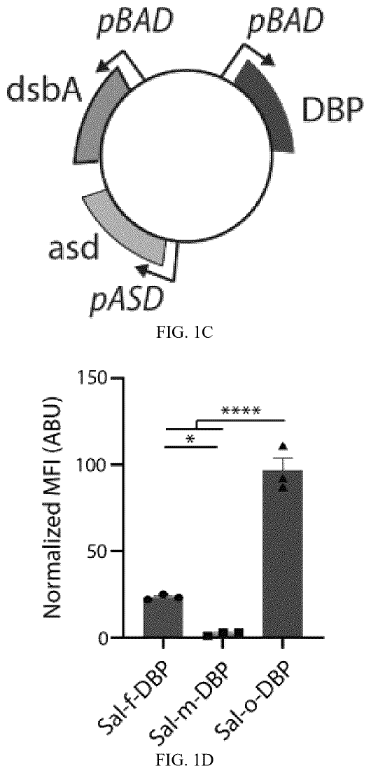

Three systems that present proteins to the outer membrane of bacterial cells were fused with the DOTA binding scFv 2D12.5 (DBP) (SEQ ID NO: 14) to test binding efficacy (FIG. 1A). The scFv was fused to the N or C terminus of the protein depending on the protein structure and was linked using three G4S1 linker regions surrounding a flag tag. The construct was then placed under the arabinose inducible promoter pBAD for controlled expression (FIG. 1B). Bacterial fimbrae are surface structures consisting of a base structure with pili that extend out from the cell surface. Fusion of 2D12.5 on the C terminus of fimH (Sal-f-DBP) displays the scFv at the end of the pili. The β-barrel of misL traffics to the outer membrane using translocator domain and features an N passenger domain to carry 2D12.5 across the outer membrane (Sal-m-DBP). The outer membrane protein OmpA is a canonical surface display molecule. The five 3-sheet domains of the protein OmpA cross the bacterial outer membrane while the Lpp motif (SEQ ID NO: 26) anchors the domain to the lipid bilayer; fusion of the 2D12.5 protein sequence to the C terminal results in presentation to the extracellular space (Sal-o-DBP). The various DOTA binding constructs (DBP) were transformed into a plasmid containing the gene asd for plasmid retention (FIG. 1C). After culture in a solution of DOTA Sal-o-DBP binds DOTA more than Sal-f-DBP (P<0.0001) or Sal-m-DBP (P<0.0001). The fusion fimH-2D12.5 bound more than Sal-m-DBP (P=0.0305) which had no visible binding (FIG. 1D).

### Sal-DBP Traffics 2D12.5 to the Periplasm

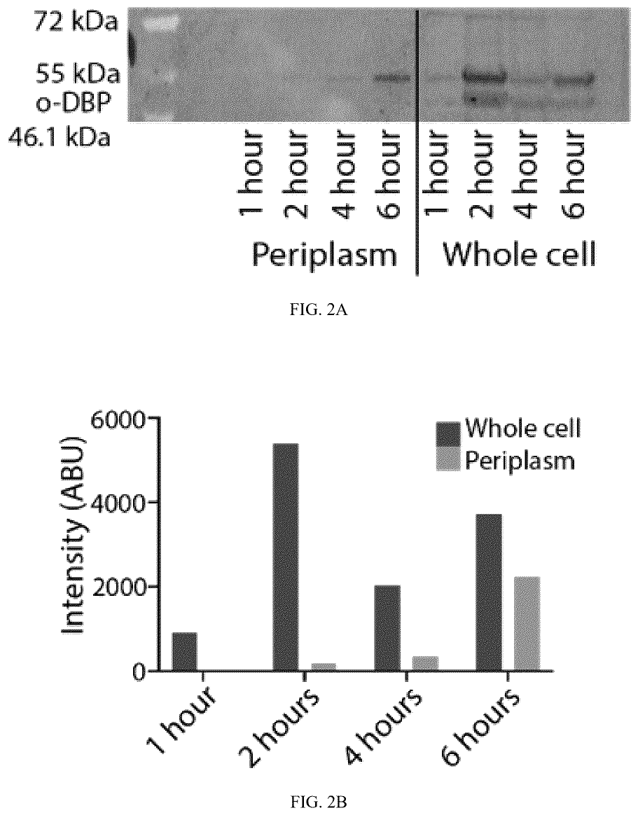

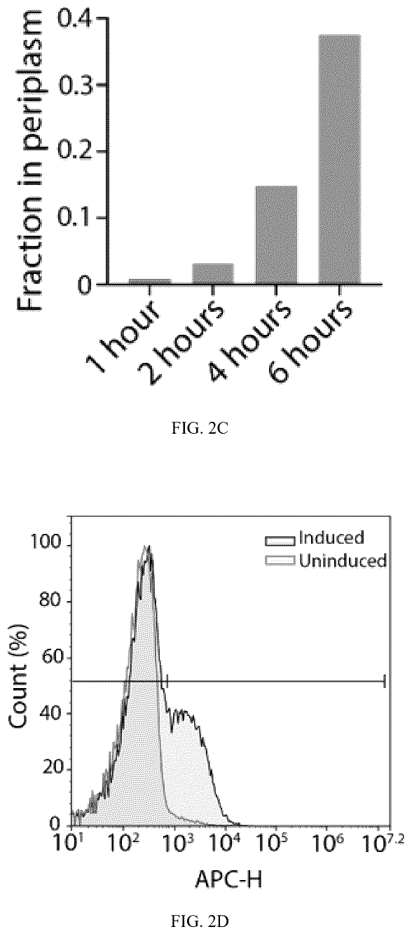

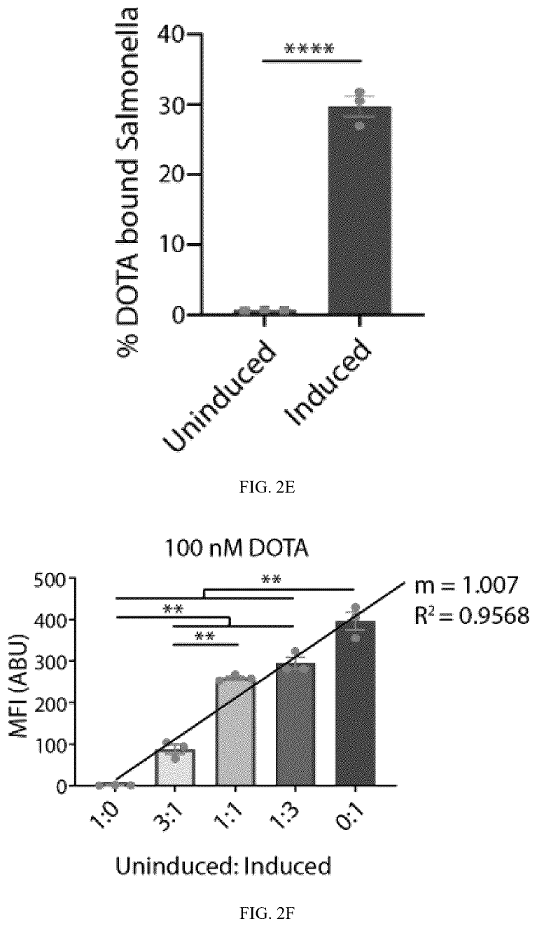

For DOTA binding to occur DBP must be presented on the outer surface of the bacteria. Additionally, scFv folding and the formation of sulfide bonds can only occur in the oxidizing environment of the Salmonella periplasm compared to the reducing environment of the cytoplasm. Protein localization to the periplasm is critical for formation of the DOTA binding pocket. When separated into the periplasmic and whole cell fractions, DBP was produced first in the cytoplasm before it was transported to the periplasm (FIGS. 2A-2C). The fusion protein ompA-2D12.5 is about 46.1 kDa. Overall production of DBP was highest two hours after induction with arabinose (FIG. 2B); however, localization to the periplasm peaked at 6 hours (FIG. 2C). At six hours, 40 percent of the total DBP in the bacteria were in the periplasmic fraction (FIG. 2C).

### Sal-o-DBP Binds DOTA In Vitro

Sal-o-DBP controllably binds to DOTA. In vitro binding assays demonstrate that induced Sal-o-DBP strongly binds DOTA. After mixing with 100 nM DOTA for 2 hours, thirty percent of induced Sal-o-DBP bind to DOTA while uninduced Sal-o-DBP have negligible binding (P<0.0001, FIGS. 2D,E). This supports that DOTA binding is highly specific to our engineered construct and does not bind to stray bacterial elements. After combining at five ratios, there was a linear relationship between the ratio of uninduced and induced Sal-o-DBP (FIG. 2E). The fluorescence intensity (MFI) of bound DOTA had a linear relationship with a slope m=1.007 and R²=0.9568. This relationship demonstrates the specificity of DOTA to Sal-o-DBP as without surface display there is no binding.

### Disulfide Bond Forming Protein Improves Sal-DBP Binding

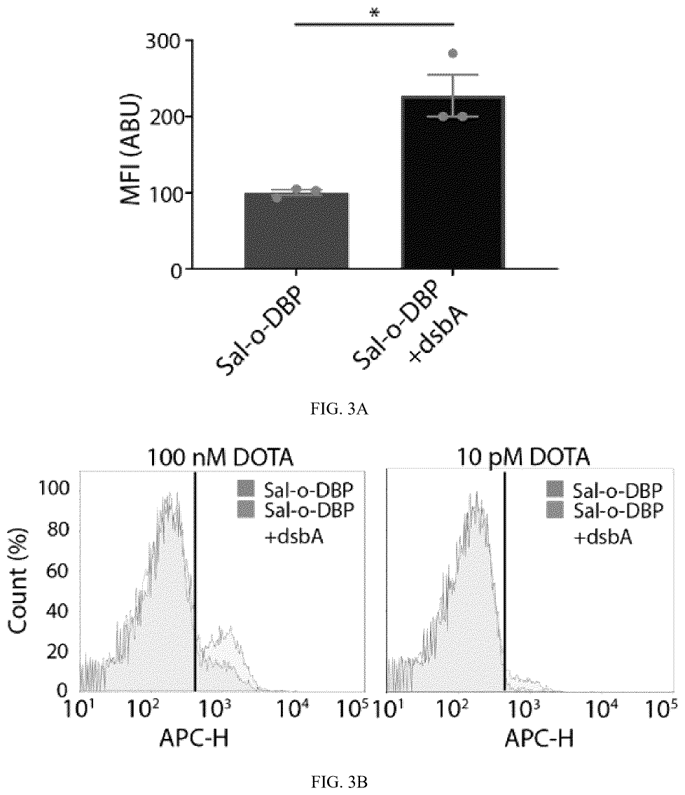

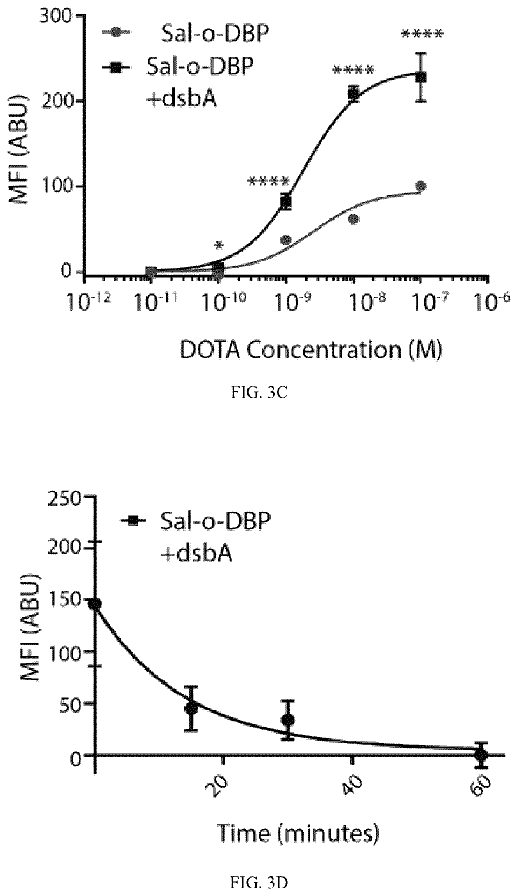

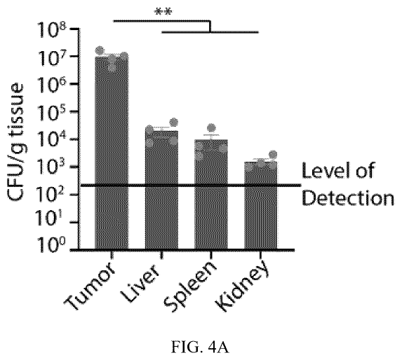

To improve the folding and binding ability of Sal-DBP, disulfide bond forming protein A (dsbA) (SEQ ID NO: 16) was added to the engineered plasmid. Adding dsbA doubled the DOTA bound Salmonella (P=0.0103; FIG. 3A). The addition of dsbA (red) shifted the bound bacterial peak to the right compared to bacteria without dsbA (blue), indicating stronger binding of the DOTA bound bacteria by stronger fluorescent signal (FIG. 3B). Adding dsbA increased the sensitivity to DOTA as well. When cultured with 10 pM of DOTA Sal-o-DBP+dsbA bound at small fractions to DOTA while Sal-o-DSB-dsbA did not (FIG. 3B). The addition of dsbA increased binding at multiple concentrations of DOTA (FIG. 3C). Sal-o-DBP+dsbA bound to more DOTA at 100 nM (P<0.0001), 10 nM (P<0.0001), 1 nM (P<0.0001), and 100 pM (P=0.0415).

Sal-o-DBP has strong binding kinetics to DOTA and inducing dsbA production improved Sal-o-DBP binding kinetics (FIG. 3C and Table 1). Sal-o-DBP bound to DOTA with an equilibrium dissociation constant (K_{D}) of 2.59+/−1.57 nM. Sal-o-DBP+dsbA had a K_{D} of 1.81+/−0.26 nM. The Bₘₐₓ of Sal-o-DBP was 95.03+/−11.74. Adding dsbA increased the Bₘₐₓ to 237.00+/−6.70. The binding kinetics of free 2D12.5 to DOTA-biotin-Y was 3.7+/−3.6 nM which demonstrates that our Salmonella based binding system has binding efficacy similar to the pure scFv. Sal-o-DBP+dsbA had a K_{off} of 0.071+/−0.031 min⁻¹ and a half-life of 9.77 min (calculated as ln(2)/K_{off}) (FIG. 3D and Table 1).

### Sal-o-DBP Localizes to and Accumulates DOTA in Tumors

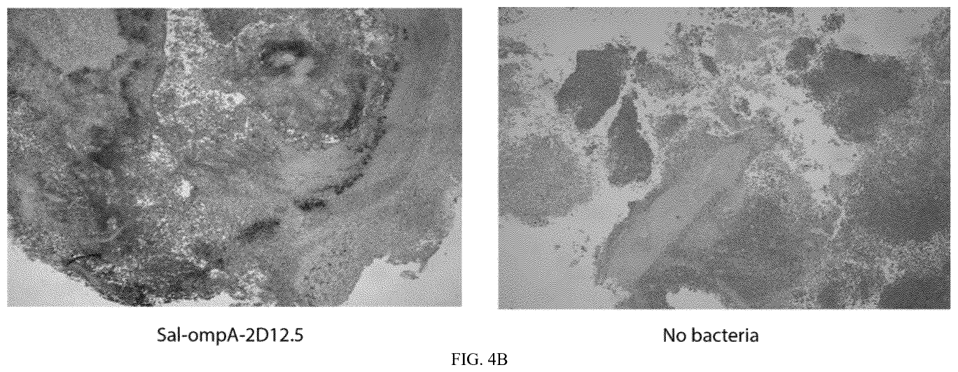

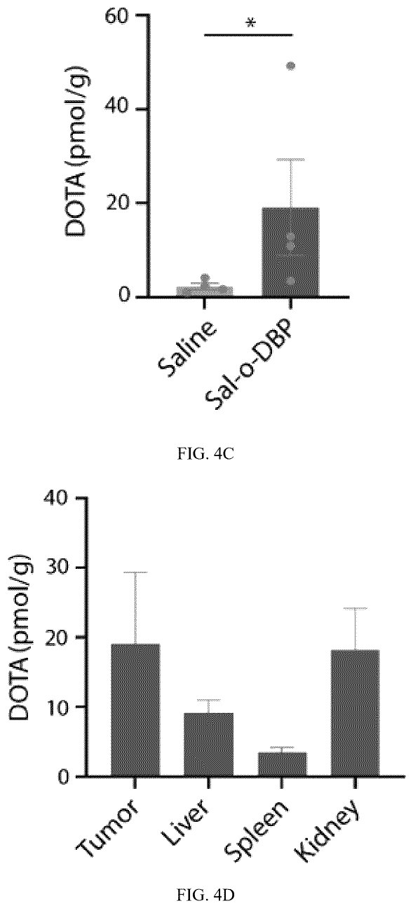

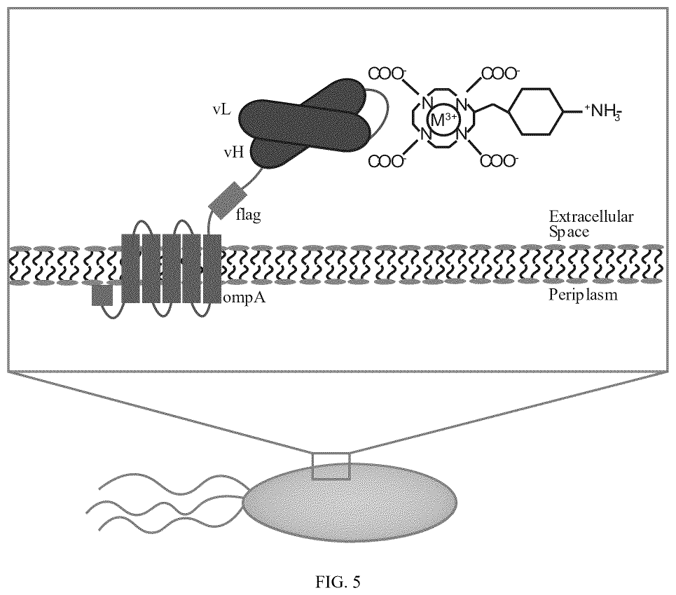

Salmonella accumulated more in tumors than livers, spleens, or kidneys (P=0.010, FIG. 4A). Histology slides of tumors show Sal-o-DBP are present throughout the tumor microenvironment after intravenous injection (brown) with lack of penetration into some dense tissue sections (FIG. 4B left). The same staining of an untreated tumor shows no bacterial density (FIG. 4B right).

Salmonella expressing DBP increased DOTA localization to tumors. Compared to mice that were not injected with bacteria, intratumoral injection of Sal-o-DBP increased DOTA accumulation in tumors (P=0.0468, FIG. 4C). Intratumoral Sal-o-DBP resulted in highly variable DOTA accumulation. Intravenous injection of Sal-o-DBP did not result in significant accumulation of DOTA (FIG. 4C), despite there being similar levels of bacterial biodistribution (FIG. 4A). Biodistribution of DOTA in the tumor, liver, spleen, and kidney from mice injected intratumorally with Sal-o-DBP was performed (FIG. 4D). Two hours after intravenous DOTA injection there was strong levels in the tumor and kidneys with some DOTA remaining in the liver and spleen.

Any patents, applications and publications as listed throughout this document are hereby incorporated by reference in their entirety herein.

### Tables

TABLE 1
Quantification of binding affinity (K_{d}), maximum binding (Bₘₐₓ),
and dissociation constant (K_{off}) for Sal-o-DBP with and without dsbA.

| | K_{D} | Bₘₐₓ | R² for | K_{off} | R² for |
|---|---|---|---|---|---|
| Strain | (nM) | (MFI) | K_{D} | (min⁻¹) | K_{off} |
| Sal-o-DBP | 2.59 +/− 1.57 | 95.03 +/− 11.74 | 0.94 | | |
| (SEQ ID NO: 28) | | | | | |
| Sal-o-DBP + dsbA | 1.81 +/− 0.26 | 237.00 +/− 6.70 | 0.99 | 0.071 +/− 0.031 | 0.98 |
| (SEQ ID NO: 29) | | | | | |

TABLE 2
Primer sequences for cloning.

| Primer | Sequence |
|---|---|
| SEQ ID NO: 30 | GCTGATACCGCTCGGAAGACTTCAGCTGGGTTGACGTTGATAGT |
| misL fwd | CTG |
| SEQ ID NO: 31 | TGCCTGGCAGTTTATGAAGACGCGCACTTAATCGTTATCAATAGC |
| misL rev | ATTGTGGC |
| SEQ ID NO: 32 | GCTGATACCGCTCGGAAGACTTGGAGGTGGCGGATCTGG |
| Flag tag fwd for | |
| misL | |
| SEQ ID NO: 33 | TGCCTGGCAGTTTATGAAGACGCGCTGCCTCCTCCACCAC |
| Flag tag rev for | |
| misL | |
| SEQ ID NO: 34 | GCTGATACCGCTCGGAAGACTTAGGATTTACACTTTATGCTTCCG |
| 2D12.5 fwd for | GCTCGTATAATGAAAAAAAAAGGAGGAAAAAAATGCAGGTGAA |
| misL | ACTGCAGGAAAG |
| SEQ ID NO: 35 | TGCCTGGCAGTTTATGAAGACGCCTCCGCCCAGCACGGTCAGG |
| 2D12.5 rev for | |
| misL | |
| SEQ ID NO: 36 | GCTGATACCGCTCGGAAGACTTAGGAGACGCTTTTTATCGCAACT |
| fimH fwd | CTCTACTGTGAATTCATTAAAGAGGAGAAAGGTCATATGAAAAT |
| | ATACTCAGCGCTATTGC |
| SEQ ID NO: 37 | TGCCTGGCAGTTTATGAAGACGCATCATAATCGACTCGTAGATA |
| fimH rev | GC |
| SEQ ID NO: 38 | GCTGATACCGCTCGGAAGACTTTGATGGAGGTGGCGGATCTGGT |
| 2D12.5 fwd | |
| (w/fimH) | |
| SEQ ID NO: 39 | TGCCTGGCAGTTTATGAAGACGCGCACTTAGCCCAGCACGGTCA |
| 2D12.5 rev | GG |
| (w/fimH) | |
| SEQ ID NO: 40 | AAGTCTTCCGAGCGGTATCAGC |
| Part plasmid bb | |
| fwd | |
| SEQ ID NO: 41 | GCGTCTTCATAAACTGCCAGGCA |
| Part plasmid bb | |
| rev | |
| SEQ ID NO: 18 | AGCTCCCGGAGACGGTCGACGCTTTTTATCGCAACTCTCTACTGT |
| dsbA fwd | AAAAAAAAAAGGAGGAAAAAAAATGAAAAAGATTTGGCTGGCG |
| | C |
| SEQ ID NO: 19 | ATCCGCTTACAGACAAGCTGTTTACAGATCCTCTTCTGAGATGAG |
| dsbA rev | TTTTTGTTCTTTTTTATCAACCAAATATTTCACAGTATC |
| SEQ ID NO: 20 | ACAGCTTGTCTGTAAGCGGAT (SEQ ID NO: 20) |
| BB dsbA fwd | |
| SEQ ID NO: 21 | GACCGTCTCCGGGAGCT (SEQ ID NO: 21) |
| BB dsbA rev | |
| SEQ ID NO: 22 | ATTTCACACCGCATATGGTGCCGTACGTTTTCGTTCCATTGG (SEQ |
| asd fwd | ID NO: 22) |
| SEQ ID NO: 23 | CAGAGCAGATTGTACTGAGAGTATCTGCGTTTACTCCTGTATTAC |
| asd rev | (SEQ ID NO: 23) |
| SEQ ID NO: 24 | ACTCTCAGTACAATCTGCTCTG (SEQ ID NO: 24) |
| BB for asd fwd | |
| SEQ ID NO: 25 | GCACCATATGCGGTGTGAAAT (SEQ ID NO: 25 |
| BB for asd rev | |

SEQUENCES

SEQ ID NO: 1 - 2D12.5 scFv
QVKLQESGPGLVQPSQSLSITCTVSGFSLTDYGVHWVRQSPGKGLEWLGVIWSGGGTA
YTAAFISRLNIYKDNSKNQVFFEMNSLQANDTAMYYCARRGSYPYNYFDVWGQGTTV
TVSSGGGGSGGGGSGGGGSQAVVTQESALTTSPGETVTLTCRSSTGAVTTSNYANWVQ
EKPDHLFTGLIGGNNNRPPGVPARFSGSLIGDKAALTIAGTQTEDEAIYFCALWYSNHW
VFGGGTRLTVLG
SEQ ID NO: 2 - 2D12.5 scFv
CAGGTGAAACTGCAGGAAAGCGGCCCGGGCCTGGTGCAGCCGAGCCAGAGCCTGA
GCATTACCTGCACCGTGAGCGGCTTTAGCCTGACCGATTATGGCGTGCATTGGGTGC
GCCAGAGCCCGGGCAAAGGCCTGGAATGGCTGGGCGTGATTTGGAGCGGCGGCGGC
ACCGCGTATACCGCGGCGTTTATTAGCCGCCTGAACATTTATAAAGATAACAGCAAA
AACCAGGTGTTTTTTGAAATGAACAGCCTGCAGGCGAACGATACCGCGATGTATTAT
TGCGCGCGCCGCGGCAGCTATCCGTATAACTATTTTGATGTGTGGGGCCAGGGCACC
ACCGTGACCGTGAGCAGCGGTGGAGGTGGTAGCGGTGGAGGAGGTAGCGGTGGAG
GTGGTAGCCAGGCGGTGGTGACCCAGGAAAGCGCGCTGACCACCAGCCCGGGCGAA
ACCGTGACCCTGACCTGCCGCAGCAGCACCGGCGCGGTGACCACCAGCAACTATGC
GAACTGGGTGCAGGAAAAACCGGATCATCTGTTTACCGGCCTGATTGGCGGCAACA
ACAACCGCCCGCCGGGCGTGCCGGCGCGCTTTAGCGGCAGCCTGATTGGCGATAAA
GCGGCGCTGACCATTGCGGGCACCCAGACCGAAGATGAAGCGATTTATTTTTGCGCG
CTGTGGTATAGCAACCATTGGGTGTTTGGCGGCGGCACCCGCCTGACCGTGCTGGGC
TAA
SEQ ID NO: 3 - ompA
NPYVGFEMGYDWLGRMPYKGDNINGAYKAQGVOLTAKLGYPITDDLDVYTRLGGMV
WRADTKSNVPGGPSTKDHDTGVSPVFAGGIEYAITPEIATRLEYQWTNNIGDANTIGTRP
DN
SEQ ID NO: 4 - ompA
AACCCGTATGTTGGCTTTGAAATGGGCTACGACTGGTTAGGCCGTATGCCGTACAAA
GGCGACAACATCAATGGCGCTTATAAAGCTCAGGGCGTTCAGTTGACCGCTAAACT
GGGTTATCCAATCACTGACGATCTGGACGTTTATACCCGTCTGGGTGGTATGGTATG
GCGTGCAGACACCAAGTCTAACGTCCCTGGCGGCCCGTCTACTAAAGACCACGACA
CCGGCGTTTCCCCGGTATTCGCGGGCGGTATCGAGTATGCTATCACCCCTGAAATCG
CAACCCGTCTGGAATACCAGTGGACTAACAACATCGGTGATGCCAACACCATCGGC
ACCCGTCCGGACAAC
SEQ ID NO: 5 - linker 1
GGGSGGGGSGGGGS
SEQ ID NO: 6 - linker 1
GGAGGTGGTAGTGGTGGAGGTGGAAGTGGTGGAGGAGGCAGC
SEQ ID NO: 7 - linker 2
GGGGSGGGGRGGGGR
SEQ ID NO: 8 - linker 2
GGAGGTGGCGGATCTGGTGGAGGTGGCAGAGGTGGAGGTGGCAGAGACTACAAAG
ACCATGACGGTGATTATAAAGATCATGACATC
SEQ ID NO: 9 - flag tag
DYKDHDGDYKDHDIDYKDDDDK
SEQ ID NO: 10 - flag tag
GATTACAAGGATGACGATGACAAG
SEQ ID NO: 11 - full linker
GGGGSGGGGRGGGGRDYKDHDGDYKDHDIDYKDDDDKGGGSGGGGSGGGGS
SEQ ID NO: 12 - full linker
GGAGGTGGCGGATCTGGTGGAGGTGGCAGAGGTGGAGGTGGCAGAGACTACAAAG
ACCATGACGGTGATTATAAAGATCATGACATCGATTACAAGGATGACGATGACAAG
GGAGGTGGTAGTGGTGGAGGTGGAAGTGGTGGAGGAGGCAGC
SEQ ID NO: 13 - 1pp-ompA-flag-2D12.5
ATGAAAGCTACTAAACTGGTACTGGGCGCGGTAATCCTGGGTTCTACTCTGCTGGCA
GGTTGCTCCAGCAACGCTAAAATCGATCAGAACCCGTATGTTGGCTTTGAAATGGGC
TACGACTGGTTAGGCCGTATGCCGTACAAAGGCGACAACATCAATGGCGCTTATAA
AGCTCAGGGCGTTCAGTTGACCGCTAAACTGGGTTATCCAATCACTGACGATCTGGA
CGTTTATACCCGTCTGGGTGGTATGGTATGGCGTGCAGACACCAAGTCTAACGTCCC
TGGCGGCCCGTCTACTAAAGACCACGACACCGGCGTTTCCCCGGTATTCGCGGGCGG
TATCGAGTATGCTATCACCCCTGAAATCGCAACCCGTCTGGAATACCAGTGGACTAA
CAACATCGGTGATGCCAACACCATCGGCACCCGTCCGGACAACGGAGGTGGCGGAT
CTGGTGGAGGTGGCAGAGGTGGAGGTGGCAGAGACTACAAAGACCATGACGGTGAT
TATAAAGATCATGACATCGATTACAAGGATGACGATGACAAGGGAGGTGGTAGTGG
TGGAGGTGGAAGTGGTGGAGGAGGCAGCCAGGTGAAACTGCAGGAAAGCGGCCCG
GGCCTGGTGCAGCCGAGCCAGAGCCTGAGCATTACCTGCACCGTGAGCGGCTTTAG
CCTGACCGATTATGGCGTGCATTGGGTGCGCCAGAGCCCGGGCAAAGGCCTGGAAT
GGCTGGGCGTGATTTGGAGCGGCGGCGGCACCGCGTATACCGCGGCGTTTATTAGCC
GCCTGAACATTTATAAAGATAACAGCAAAAACCAGGTGTTTTTTGAAATGAACAGC
CTGCAGGCGAACGATACCGCGATGTATTATTGCGCGCGCCGCGGCAGCTATCCGTAT
AACTATTTTGATGTGTGGGGCCAGGGCACCACCGTGACCGTGAGCAGCGGTGGAGG
TGGTAGCGGTGGAGGAGGTAGCGGTGGAGGTGGTAGCCAGGCGGTGGTGACCCAGG
AAAGCGCGCTGACCACCAGCCCGGGCGAAACCGTGACCCTGACCTGCCGCAGCAGC
ACCGGCGCGGTGACCACCAGCAACTATGCGAACTGGGTGCAGGAAAAACCGGATCA
TCTGTTTACCGGCCTGATTGGCGGCAACAACAACCGCCCGCCGGGCGTGCCGGCGC
GCTTTAGCGGCAGCCTGATTGGCGATAAAGCGGCGCTGACCATTGCGGGCACCCAG
ACCGAAGATGAAGCGATTTATTTTTGCGCGCTGTGGTATAGCAACCATTGGGTGTTT
GGCGGCGGCACCCGCCTGACCGTGCTGGGCTAA
SEQ ID NO: 14 - 1pp-ompA-flag-2D12.5
MKATKLVLGAVILGSTLLAGCSSNAKIDQNPYVGFEMGYDWLGRMPYKGDNINGAYK
AQGVQLTAKLGYPITDDLDVYTRLGGMVWRADTKSNVPGGPSTKDHDTGVSPVFAGGI
EYAITPEIATRLEYQWTNNIGDANTIGTRPDNGGGGSGGGGRGGGGRDYKDHDGDYKD
HDIDYKDDDDKGGGSGGGGSGGGGSQVKLQESGPGLVQPSQSLSITCTVSGFSLTDYGV
HWVRQSPGKGLEWLGVIWSGGGTAYTAAFISRLNIYKDNSKNQVFFEMNSLQANDTA
MYYCARRGSYPYNYFDVWGQGTTVTVSSGGGGSGGGGSGGGGSQAVVTQESALTTSP
GETVTLTCRSSTGAVTTSNYANWVQEKPDHLFTGLIGGNNNRPPGVPARFSGSLIGDKA
ALTIAGTQTEDEAIYFCALWYSNHWVFGGGTRLTVLG
SEQ ID NO: 15 - dsbA
ATGAAAAAGATTTGGCTGGCGCTGGCTGGTATGGTTTTAGCTTTTAGCGCCTCGGCA
GCACAGATCAGCGACGGTAAACAGTATATCACGCTGGATAAACCGGTCGCTGGCGA
ACCCCAGGTACTGGAGTTTTTCTCCTTCTACTGCCCACATTGTTATCAGTTTGAAGAA
GTGCTTCATGTGTCTGACAATGTGAAGAAAAAGCTGCCGGAAGGCACCAAAATGAC
CAAGTACCACGTTGAGTTCCTGGGGCCGTTGGGCAAGGAGCTCACCCAGGCATGGG
CGGTGGCGATGGCGTTGGGTGTAGAAGATAAAGTCACGGTCCCGCTGTTTGAAGCC
GTACAGAAAACCCAGACAGTACAATCTGCCGCGGATATCCGTAAAGTGTTCGTTGAT
GCGGGCGTCAAGGGCGAAGATTACGATGCGGCATGGAACAGCTTCGTGGTGAAATC
ACTGGTTGCGCAACAGGAGAAAGCCGCGGCTGACCTGCAACTGCAGGGCGTTCCGG
CGATGTTCGTCAATGGCAAATACCAGATTAACCCACAAGGCATGGATACGAGCAGC
ATGGATGTTTTTGTTCAGCAGTATGCTGATACTGTGAAATATTTGGTTGATAAAAAA
GAACAAAAACTCATCTCAGAAGAGGATCTG
SEQ ID NO: 16 - dsbA
MKKIWLALAGMVLAFSASAAQISDGKQYITLDKPVAGEPQVLEFFSFYCPHCYQFEEVL
HVSDNVKKKLPEGTKMTKYHVEFLGPLGKELTQAWAVAMALGVEDKVTVPLFEAVQ
KTQTVQSAADIRKVFVDAGVKGEDYDAAWNSFVVKSLVAQQEKAAADLQLQGVPAM
FVNGKYQINPQGMDTSSMDVFVQQYADTVKYLVDKKEQKLISEEDL
SEQ ID NO: 17 - asd
ATGGTGAAGGATGCGCCACAGGATACTGGCGCGCATACACAGCACATCTCTTTGCA
GGAAAAAAACGCTATGAAAAATGTTGGTTTTATCGGCTGGCGCGGAATGGTCGGCT
CTGTTCTCATGCAACGCATGGTAGAGGAGCGCGATTTCGACGCTATTCGCCCTGTTT
TCTTTTCTACCTCCCAGTTTGGACAGGCGGCGCCCACCTTCGGCGACACCTCCGGCA
CGCTACAGGACGCTTTTGACCTGGATGCGCTAAAAGCGCTCGATATCATCGTGACCT
GCCAGGGCGGCGATTATACCAACGAAATTTATCCAAAGCTGCGCGAAAGCGGATGG
CAGGGTTACTGGATTGATGCGGCTTCTACGCTGCGCATGAAAGATGATGCCATTATT
ATTCTCGACCCGGTCAACCAGGACGTGATTACCGACGGCCTGAACAATGGCGTGAA
AACCTTTGTGGGCGGTAACTGTACCGTTAGCCTGATGTTGATGTCGCTGGGCGGTCT
GTTTGCCCATAATCTCGTTGACTGGGTATCCGTTGCAACTTATCAAGCAGCTTCTGGT
GGTGGTGCACGTCATATGCGTGAGCTGTTAACCCAGATGGGTCAGTTGTATGGCCAT
GTCGCCGATGAACTGGCGACGCCGAGCTCCGCAATTCTTGATATTGAACGCAAAGTT
ACGGCATTGACCCGCAGCGGCGAGCTGCCGGTTGATAACTTTGGCGTACCGCTGGC
GGGAAGCCTGATCCCCTGGATCGACAAACAGCTCGATAACGGCCAGAGCCGCGAAG
AGTGGAAAGGCCAGGCGGAAACCAACAAGATTCTCAATACTGCCTCTGTGATTCCG
GTTGATGGTTTGTGTGTGCGCGTCGGCGCGCTGCGCTGTCACAGCCAGGCGTTCACC
ATCAAGCTGAAAAAAGAGGTATCCATTCCGACGGTGGAAGAACTGCTGGCGGCACA
TAATCCGTGGGCGAAAGTGGTGCCGAACGATCGTGATATCACTATGCGCGAATTAA
CCCCGGCGGCGGTGACCGGCACGTTGACTACGCCGGTTGGTCGTCTGCGTAAGCTGA
ACATGGGGCCAGAGTTCTTGTCGGCGTTTACCGTAGGCGACCAGTTGTTATGGGGCG
CCGCCGAGCCGCTGCGTCGAATGCTGCGCCAGTTGGCGTAG
SEQ ID NO: 18 - primer
AGCTCCCGGAGACGGTCGACGCTTTTTATCGCAACTCTCTACTGTAAAAAAAAAAGG
AGGAAAAAAAATGAAAAAGATTTGGCTGGCGC
SEQ ID NO: 19 - primer
ATCCGCTTACAGACAAGCTGTTTACAGATCCTCTTCTGAGATGAGTTTTTGTTCTTTT
TTATCAACCAAATATTTCACAGTATC
SEQ ID NO: 20 - primer
ACAGCTTGTCTGTAAGCGGAT
SEQ ID NO: 21 - primer
GACCGTCTCCGGGAGCT
SEQ ID NO: 22 - primer
ATTTCACACCGCATATGGTGCCGTACGTTTTCGTTCCATTGG
SEQ ID NO: 23 - primer
CAGAGCAGATTGTACTGAGAGTATCTGCGTTTACTCCTGTATTAC
SEQ ID NO: 24 - primer
ACTCTCAGTACAATCTGCTCTG
SEQ ID NO: 25 - primer
GCACCATATGCGGTGTGAAAT
SEQ ID NO: 26 - 1pp
MKATKLVLGAVILGSTLLAGCSSNAKIDQ
SEQ ID NO: 27 - 1pp
ATGAAAGCTACTAAACTGGTACTGGGCGCGGTAATCCTGGGTTCTACTCTGCTGGCA
GGTTGCTCCAGCAACGCTAAAAT
SEQ ID NO: 28 - full plasmid without dsbA
ATGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTCCC
GGCAACAATTAATAGACTGGATGGAGGCGGATAAAGTTGCAGGACCACTTCTGCGC
TCGGCCCTTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTATCGTAGTT
ATCTACACGACGGGGAGTCAGGCAACTATGGATGAACGAAATAGACAGATCGCTGA
GATAGGTGCCTCACTGATTAAGCATTGGTAACTGTCAGACCAAGTTTACTCATATAT
ACTTTAGATTGATTTAAAACTTCATTTTTAATTTAAAAGGATCTAGGTGAAGATCCTT
TTTGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAGAA
AGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTC
TTTCCTGCGTTATCCCCTGATTCTGTGGATAACCGTATTACCGCCTTTGAGTGAGCTG
ATACCGCTCGCCGCAGCCGAACGACCGAGCGCAGCGAGTCAGTGAGCGAGGAAGC
GGAAGAGCGCCTGATGCGGTATTTTCTCCTTACGCATCTGTGCGGTATTTCACACCG
CATATGGTGCACTCTCAGTACAATCTGCTCTGATGCCGCATAGTTAAGCCAGTATAC
ACTCCGCTATCGCTACGTGACTGGGTCATGGCTGCGCCCCGACACCCGCCAACACCC
GCTGACGCGCCCTGACGGGCTTGTCTGCTCCCGGCATCCGCTTACAGACAAGCTGTG
ACCGTCTCCGGGAGCTGCATGTGTCAGAGGTTTTCACCGTCATCACCGAAACGCGCG
AGGCAGCAGATCAATTCGCGCGCGAAGGCGAAGCGGCATGCATAATGTGCCTGTCA
AATGGACGAAGCAGGGATTCTGCAAACCCTATGCTACTCCGTCAAGCCGTCAATTGT
CTGATTCGTTACCAATTATGACAACTTGACGGCTACATCATTCACTTTTTCTTCACAA
CCGGCACGGAACTCGCTCGGGCTGGCCCCGGTGCATTTTTTAAATACCCGCGAGAAA
TAGAGTTGATCGTCAAAACCAACATTGCGACCGACGGTGGCGATAGGCATCCGGGT
GGTGCTCAAAAGCAGCTTCGCCTGGCTGATACGTTGGTCCTCGCGCCAGCTTAAGAC
GCTAATCCCTAACTGCTGGCGGAAAAGATGTGACAGACGCGACGGCGACAAGCAAA
CATGCTGTGCGACGCTGGCGATATCAAAATTGCTGTCTGCCAGGTGATCGCTGATGT
ACTGACAAGCCTCGCGTACCCGATTATCCATCGGTGGATGGAGCGACTCGTTAATCG
CTTCCATGCGCCGCAGTAACAATTGCTCAAGCAGATTTATCGCCAGCAGCTCCGAAT
AGCGCCCTTCCCCTTGCCCGGCGTTAATGATTTGCCCAAACAGGTCGCTGAAATGCG
GCTGGTGCGCTTCATCCGGGCGAAAGAACCCCGTATTGGCAAATATTGACGGCCAGT
TAAGCCATTCATGCCAGTAGGCGCGCGGACGAAAGTAAACCCACTGGTGATACCAT
TCGCGAGCCTCCGGATGACGACCGTAGTGATGAATCTCTCCTGGCGGGAACAGCAA
AATATCACCCGGTCGGCAAACAAATTCTCGTCCCTGATTTTTCACCACCCCCTGACC
GCGAATGGTGAGATTGAGAATATAACCTTTCATTCCCAGCGGTCGGTCGATAAAAA
AATCGAGATAACCGTTGGCCTCAATCGGCGTTAAACCCGCCACCAGATGGGCATTA
AACGAGTATCCCGGCAGCAGGGGATCATTTTGCGCTTCAGCCATACTTTTCATACTC
CCGCCATTCAGAGAAGAAACCAATTGTCCATATTGCATCAGACATTGCCGTCACTGC
GTCTTTTACTGGCTCTTCTCGCTAACCAAACCGGTAACCCCGCTTATTAAAAGCATTC
TGTAACAAAGCGGGACCAAAGCCATGACAAAAACGCGTAACAAAAGTGTCTATAAT
CACGGCAGAAAAGTCCACATTGATTATTTGCACGGCGTCACACTTTGCTATGCCATA
GCATTTTTATCCATAAGATTAGCGGATCCTACCTGACGCTTTTTATCGCAACTCTCTA
CTGTTTCTCCATACCCGTTTTTTGGGCTAACAGGAGGAATTAACCATGAAAGCTACT
AAACTGGTACTGGGCGCGGTAATCCTGGGTTCTACTCTGCTGGCAGGTTGCTCCAGC
AACGCTAAAATCGATCAGAACCCGTATGTTGGCTTTGAAATGGGCTACGACTGGTTA
GGCCGTATGCCGTACAAAGGCGACAACATCAATGGCGCTTATAAAGCTCAGGGCGT
TCAGTTGACCGCTAAACTGGGTTATCCAATCACTGACGATCTGGACGTTTATACCCG
TCTGGGTGGTATGGTATGGCGTGCAGACACCAAGTCTAACGTCCCTGGCGGCCCGTC
TACTAAAGACCACGACACCGGCGTTTCCCCGGTATTCGCGGGCGGTATCGAGTATGC
TATCACCCCTGAAATCGCAACCCGTCTGGAATACCAGTGGACTAACAACATCGGTG
ATGCCAACACCATCGGCACCCGTCCGGACAACGGAGGTGGCGGATCTGGTGGAGGT
GGCAGAGGTGGAGGTGGCAGAGACTACAAAGACCATGACGGTGATTATAAAGATCA
TGACATCGATTACAAGGATGACGATGACAAGGGAGGTGGTAGTGGTGGAGGTGGAA
GTGGTGGAGGAGGCAGCCAGGTGAAACTGCAGGAAAGCGGCCCGGGCCTGGTGCA
GCCGAGCCAGAGCCTGAGCATTACCTGCACCGTGAGCGGCTTTAGCCTGACCGATTA
TGGCGTGCATTGGGTGCGCCAGAGCCCGGGCAAAGGCCTGGAATGGCTGGGCGTGA
TTTGGAGCGGCGGCGGCACCGCGTATACCGCGGCGTTTATTAGCCGCCTGAACATTT
ATAAAGATAACAGCAAAAACCAGGTGTTTTTTGAAATGAACAGCCTGCAGGCGAAC
GATACCGCGATGTATTATTGCGCGCGCCGCGGCAGCTATCCGTATAACTATTTTGAT
GTGTGGGGCCAGGGCACCACCGTGACCGTGAGCAGCGGTGGAGGTGGTAGCGGTGG
AGGAGGTAGCGGTGGAGGTGGTAGCCAGGCGGTGGTGACCCAGGAAAGCGCGCTG
ACCACCAGCCCGGGCGAAACCGTGACCCTGACCTGCCGCAGCAGCACCGGCGCGGT
GACCACCAGCAACTATGCGAACTGGGTGCAGGAAAAACCGGATCATCTGTTTACCG
GCCTGATTGGCGGCAACAACAACCGCCCGCCGGGCGTGCCGGCGCGCTTTAGCGGC
AGCCTGATTGGCGATAAAGCGGCGCTGACCATTGCGGGCACCCAGACCGAAGATGA
AGCGATTTATTTTTGCGCGCTGTGGTATAGCAACCATTGGGTGTTTGGCGGCGGCAC
CCGCCTGACCGTGCTGGGCTAAATGGATCCGAGCTCGAGATCTGCAGCTGGTACCAT
ATGGGAATTCGAAGCTTGGGCCCGAACAAAAACTCATCTCAGAAGAGGATCTGAAT
AGCGCCGTCGACCATCATCATCATCATCATTGAGTTTAAACGGTCTCCAGCTTGGCT
GTTTTGGCGGATGAGAGAAGATTTTCAGCCTGATACAGATTAAATCAGAACGCAGA
AGCGGTCTGATAAAACAGAATTTGCCTGGCGGCAGTAGCGCGGTGGTCCCACCTGA
CCCCATGCCGAACTCAGAAGTGAAACGCCGTAGCGCCGATGGTAGTGTGGGGTCTC
CCCATGCGAGAGTAGGGAACTGCCAGGCATCAAATAAAACGAAAGGCTCAGTCGAA
AGACTGGGCCTTTCGTTTTATCTGTTGTTTGTCGGTGAACGCTCTCCTGAGTAGGACA
AATCCGCCGGGAGCGGATTTGAACGTTGCGAAGCAACGGCCCGGAGGGTGGCGGGC
AGGACGCCCGCCATAAACTGCCAGGCATCAAATTAAGCAGAAGGCCATCCTGACGG
ATGGCCTTTTTGCGTTTCTACAAACTCTTTTGTTTATTTTTCTAAATACATTCAAATAT
GTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTG
CCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTATGT
GGCGCGGTATTATCCCGTGTTGACGCCGGGCAAGAGCAACTCGGTCGCCGCATACA
CTATTCTCAGAATGACTTGGTTGAGTACTCACCAGTCACAGAAAAGCATCTTACGGA
TGGCATGACAGTAAGAGAATTATGCAGTGCTGCCATAACC
SEQ ID NO: 29 - full plasmid with dsbA
ACAGTAGAGAGTTGCGATAAAAAGCGTCGACCGTCTCCGGGAGCTGCATGTGTCAG
AGGTTTTCACCGTCATCACCGAAACGCGCGAGGCAGCAGATCAATTCGCGCGCGAA
GGCGAAGCGGCATGCATAATGTGCCTGTCAAATGGACGAAGCAGGGATTCTGCAAA
CCCTATGCTACTCCGTCAAGCCGTCAATTGTCTGATTCGTTACCAATTATGACAACTT
GACGGCTACATCATTCACTTTTTCTTCACAACCGGCACGGAACTCGCTCGGGCTGGC
CCCGGTGCATTTTTTAAATACCCGCGAGAAATAGAGTTGATCGTCAAAACCAACATT
GCGACCGACGGTGGCGATAGGCATCCGGGTGGTGCTCAAAAGCAGCTTCGCCTGGC
TGATACGTTGGTCCTCGCGCCAGCTTAAGACGCTAATCCCTAACTGCTGGCGGAAAA
GATGTGACAGACGCGACGGCGACAAGCAAACATGCTGTGCGACGCTGGCGATATCA
AAATTGCTGTCTGCCAGGTGATCGCTGATGTACTGACAAGCCTCGCGTACCCGATTA
TCCATCGGTGGATGGAGCGACTCGTTAATCGCTTCCATGCGCCGCAGTAACAATTGC
TCAAGCAGATTTATCGCCAGCAGCTCCGAATAGCGCCCTTCCCCTTGCCCGGCGTTA
ATGATTTGCCCAAACAGGTCGCTGAAATGCGGCTGGTGCGCTTCATCCGGGCGAAA
GAACCCCGTATTGGCAAATATTGACGGCCAGTTAAGCCATTCATGCCAGTAGGCGCG
CGGACGAAAGTAAACCCACTGGTGATACCATTCGCGAGCCTCCGGATGACGACCGT
AGTGATGAATCTCTCCTGGCGGGAACAGCAAAATATCACCCGGTCGGCAAACAAAT
TCTCGTCCCTGATTTTTCACCACCCCCTGACCGCGAATGGTGAGATTGAGAATATAA
CCTTTCATTCCCAGCGGTCGGTCGATAAAAAAATCGAGATAACCGTTGGCCTCAATC
GGCGTTAAACCCGCCACCAGATGGGCATTAAACGAGTATCCCGGCAGCAGGGGATC
ATTTTGCGCTTCAGCCATACTTTTCATACTCCCGCCATTCAGAGAAGAAACCAATTGT
CCATATTGCATCAGACATTGCCGTCACTGCGTCTTTTACTGGCTCTTCTCGCTAACCA
AACCGGTAACCCCGCTTATTAAAAGCATTCTGTAACAAAGCGGGACCAAAGCCATG
ACAAAAACGCGTAACAAAAGTGTCTATAATCACGGCAGAAAAGTCCACATTGATTA
TTTGCACGGCGTCACACTTTGCTATGCCATAGCATTTTTATCCATAAGATTAGCGGAT
CCTACCTGACGCTTTTTATCGCAACTCTCTACTGTTTCTCCATACCCGTTTTTTGGGCT
AACAGGAGGAATTAACCATGAAAGCTACTAAACTGGTACTGGGCGCGGTAATCCTG
GGTTCTACTCTGCTGGCAGGTTGCTCCAGCAACGCTAAAATCGATCAGAACCCGTAT
GTTGGCTTTGAAATGGGCTACGACTGGTTAGGCCGTATGCCGTACAAAGGCGACAA
CATCAATGGCGCTTATAAAGCTCAGGGCGTTCAGTTGACCGCTAAACTGGGTTATCC
AATCACTGACGATCTGGACGTTTATACCCGTCTGGGTGGTATGGTATGGCGTGCAGA
CACCAAGTCTAACGTCCCTGGCGGCCCGTCTACTAAAGACCACGACACCGGCGTTTC
CCCGGTATTCGCGGGCGGTATCGAGTATGCTATCACCCCTGAAATCGCAACCCGTCT
GGAATACCAGTGGACTAACAACATCGGTGATGCCAACACCATCGGCACCCGTCCGG
ACAACGGAGGTGGCGGATCTGGTGGAGGTGGCAGAGGTGGAGGTGGCAGAGACTA
CAAAGACCATGACGGTGATTATAAAGATCATGACATCGATTACAAGGATGACGATG
ACAAGGGAGGTGGTAGTGGTGGAGGTGGAAGTGGTGGAGGAGGCAGCCAGGTGAA
ACTGCAGGAAAGCGGCCCGGGCCTGGTGCAGCCGAGCCAGAGCCTGAGCATTACCT
GCACCGTGAGCGGCTTTAGCCTGACCGATTATGGCGTGCATTGGGTGCGCCAGAGCC
CGGGCAAAGGCCTGGAATGGCTGGGCGTGATTTGGAGCGGCGGCGGCACCGCGTAT
ACCGCGGCGTTTATTAGCCGCCTGAACATTTATAAAGATAACAGCAAAAACCAGGT
GTTTTTTGAAATGAACAGCCTGCAGGCGAACGATACCGCGATGTATTATTGCGCGCG
CCGCGGCAGCTATCCGTATAACTATTTTGATGTGTGGGGCCAGGGCACCACCGTGAC
CGTGAGCAGCGGTGGAGGTGGTAGCGGTGGAGGAGGTAGCGGTGGAGGTGGTAGC
CAGGCGGTGGTGACCCAGGAAAGCGCGCTGACCACCAGCCCGGGCGAAACCGTGAC
CCTGACCTGCCGCAGCAGCACCGGCGCGGTGACCACCAGCAACTATGCGAACTGGG
TGCAGGAAAAACCGGATCATCTGTTTACCGGCCTGATTGGCGGCAACAACAACCGC
CCGCCGGGCGTGCCGGCGCGCTTTAGCGGCAGCCTGATTGGCGATAAAGCGGCGCT
GACCATTGCGGGCACCCAGACCGAAGATGAAGCGATTTATTTTTGCGCGCTGTGGTA
TAGCAACCATTGGGTGTTTGGCGGCGGCACCCGCCTGACCGTGCTGGGCTAAATGGA
TCCGAGCTCGAGATCTGCAGCTGGTACCATATGGGAATTCGAAGCTTGGGCCCGAAC
AAAAACTCATCTCAGAAGAGGATCTGAATAGCGCCGTCGACCATCATCATCATCATC
ATTGAGTTTAAACGGTCTCCAGCTTGGCTGTTTTGGCGGATGAGAGAAGATTTTCAG
CCTGATACAGATTAAATCAGAACGCAGAAGCGGTCTGATAAAACAGAATTTGCCTG
GCGGCAGTAGCGCGGTGGTCCCACCTGACCCCATGCCGAACTCAGAAGTGAAACGC
CGTAGCGCCGATGGTAGTGTGGGGTCTCCCCATGCGAGAGTAGGGAACTGCCAGGC
ATCAAATAAAACGAAAGGCTCAGTCGAAAGACTGGGCCTTTCGTTTTATCTGTTGTT
TGTCGGTGAACGCTCTCCTGAGTAGGACAAATCCGCCGGGAGCGGATTTGAACGTTG
CGAAGCAACGGCCCGGAGGGTGGCGGGCAGGACGCCCGCCATAAACTGCCAGGCA
TCAAATTAAGCAGAAGGCCATCCTGACGGATGGCCTTTTTGCGTTTCTACAAACTCT
TTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGT
CGCCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCGAA
CTGGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTGTTGACGCC
GGGCAAGAGCAACTCGGTCGCCGCATACACTATTCTCAGAATGACTTGGTTGAGTAC
TCACCAGTCACAGAAAAGCATCTTACGGATGGCATGACAGTAAGAGAATTATGCAG
TGCTGCCATAACCATGAGTGATAACACTGCGGCCAACTTACTTCTGACAACGATCGG
AGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCA
CGATGCCTGTAGCAATGGCAACAACGTTGCGCAAACTATTAACTGGCGAACTACTTA
CTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAGCCCTCCC
GTATCGTAGTTATCTACACGACGGGGAGTCAGGCAACTATGGATGAACGAAATAGA
CAGATCGCTGAGATAGGTGCCTCACTGATTAAGCATTGGTAACTGTCAGACCAAGTT
TACTCATATATACTTTAGATTGATTTAAAACTTCATTTTTAATTTAAAAGGATCTAGG
TGAAGATCCTTTTTGATAATCTCATGACCAAAATCCCTTAACGTGAGTTTTCGTTCCA
CTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCGCA
GATACCAAATACTGTCCTTCTAGTGTAGCCGTAGTTAGGCCACCACTTCAAGAACTC
TGTAGCACCGCCTACATACCTCGCTCTGCTAATCCTGTTACCAGTGGCTGCTGCCAG
TGGCGATAAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTG
CTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGA
GCGAGGAAGCGGAAGAGCGCCTGATGCGGTATTTTCTCCTTACGCATCTGTGCGGTA
TTTCACACCGCATATGGTGCCGTACGTTTTCGTTCCATTGGCCCTCAAACCCCTAATT
AGGATCAATAAAACAGCGACGGAAATGATTCCCTTCCTAACGCAAATTCCCTGATA
ATCGCCACTGGACTTTCTGCTTGCGCGGTAAGGCAGGATAAGTCGCATTACTGATGG
CTTCGCTATCATTGATTAATTTCACTTGCGACTTTGGCTGCTTTTTGTATGGTGAAGG
ATGCGCCACAGGATACTGGCGCGCATACACAGCACATCTCTTTGCAGGAAAAAAAC
GCTATGAAAAATGTTGGTTTTATCGGCTGGCGCGGAATGGTCGGCTCTGTTCTCATG
CAACGCATGGTAGAGGAGCGCGATTTCGACGCTATTCGCCCTGTTTTCTTTTCTACCT
CCCAGTTTGGACAGGCGGCGCCCACCTTCGGCGACACCTCCGGCACGCTACAGGAC
GCTTTTGACCTGGATGCGCTAAAAGCGCTCGATATCATCGTGACCTGCCAGGGCGGC
GATTATACCAACGAAATTTATCCAAAGCTGCGCGAAAGCGGATGGCAGGGTTACTG
GATTGATGCGGCTTCTACGCTGCGCATGAAAGATGATGCCATTATTATTCTCGACCC
GGTCAACCAGGACGTGATTACCGACGGCCTGAACAATGGCGTGAAAACCTTTGTGG
GCGGTAACTGTACCGTTAGCCTGATGTTGATGTCGCTGGGCGGTCTGTTTGCCCATA
ATCTCGTTGACTGGGTATCCGTTGCAACTTATCAAGCAGCTTCTGGTGGTGGTGCAC
GTCATATGCGTGAGCTGTTAACCCAGATGGGTCAGTTGTATGGCCATGTCGCCGATG
AACTGGCGACGCCGAGCTCCGCAATTCTTGATATTGAACGCAAAGTTACGGCATTGA
CCCGCAGCGGCGAGCTGCCGGTTGATAACTTTGGCGTACCGCTGGCGGGAAGCCTG
ATCCCCTGGATCGACAAACAGCTCGATAACGGCCAGAGCCGCGAAGAGTGGAAAGG
CCAGGCGGAAACCAACAAGATTCTCAATACTGCCTCTGTGATTCCGGTTGATGGTTT
GTGTGTGCGCGTCGGCGCGCTGCGCTGTCACAGCCAGGCGTTCACCATCAAGCTGAA
AAAAGAGGTATCCATTCCGACGGTGGAAGAACTGCTGGCGGCACATAATCCGTGGG
CGAAAGTGGTGCCGAACGATCGTGATATCACTATGCGCGAATTAACCCCGGCGGCG
GTGACCGGCACGTTGACTACGCCGGTTGGTCGTCTGCGTAAGCTGAACATGGGGCCA
GAGTTCTTGTCGGCGTTTACCGTAGGCGACCAGTTGTTATGGGGCGCCGCCGAGCCG
CTGCGTCGAATGCTGCGCCAGTTGGCGTAGTGGCTATTGCAGCGCTTATCGGGCCTG
CGTGTGGTTCTGTAGGCCGGATAAGGCGCGTCAGCGCCGCCATCCGGCGGGGAAAT
TTGTGTTAAACCAGGGGTGCATCGTCACCCTTTTTTTGCGTAATACAGGAGTAAACG
CAGATACTCTCAGTACAATCTGCTCTGATGCCGCATAGTTAAGCCAGTATACACTCC
GCTATCGCTACGTGACTGGGTCATGGCTGCGCCCCGACACCCGCCAACACCCGCTGA
CGCGCCCTGACGGGCTTGTCTGCTCCCGGCATCCGCTTACAGACAAGCTGTTTACAG
ATCCTCTTCTGAGATGAGTTTTTGTTCTTTTTTATCAACCAAATATTTCACAGTATCA
GCATACTGCTGAACAAAAACATCCATGCTGCTCGTATCCATGCCTTGTGGGTTAATC
TGGTATTTGCCATTGACGAACATCGCCGGAACGCCCTGCAGTTGCAGGTCAGCCGCG
GCTTTCTCCTGTTGCGCAACCAGTGATTTCACCACGAAGCTGTTCCATGCCGCATCGT
AATCTTCGCCCTTGACGCCCGCATCAACGAACACTTTACGGATATCCGCGGCAGATT
GTACTGTCTGGGTTTTCTGTACGGCTTCAAACAGCGGGACCGTGACTTTATCTTCTAC
ACCCAACGCCATCGCCACCGCCCATGCCTGGGTGAGCTCCTTGCCCAACGGCCCCAG
GAACTCAACGTGGTACTTGGTCATTTTGGTGCCTTCCGGCAGCTTTTTCTTCACATTG
TCAGACACATGAAGCACTTCTTCAAACTGATAACAATGTGGGCAGTAGAAGGAGAA
AAACTCCAGTACCTGGGGTTCGCCAGCGACCGGTTTATCCAGCGTGATATACTGTTT
ACCGTCGCTGATCTGTGCTGCCGAGGCGCTAAAAGCTAAAACCATACCAGCCAGCG
CCAGCCAAATCTTTTTCATTTTTTTTCCTCCTTTTTTTTTT
SEQ ID NO: 30 - primer
GCTGATACCGCTCGGAAGACTTCAGCTGGGTTGACGTTGATAGTCTG
SEQ ID NO: 31 - primer
TGCCTGGCAGTTTATGAAGACGCGCACTTAATCGTTATCAATAGCATTGTGGC
SEQ ID NO: 32 - primer
GCTGATACCGCTCGGAAGACTTGGAGGTGGCGGATCTGG
SEQ ID NO: 33 - primer
TGCCTGGCAGTTTATGAAGACGCGCTGCCTCCTCCACCAC
SEQ ID NO: 34 - primer
GCTGATACCGCTCGGAAGACTTAGGATTTACACTTTATGCTTCCGGCTCGTATAATG
AAAAAAAAAGGAGGAAAAAAATGCAGGTGAAACTGCAGGAAAG
SEQ ID NO: 35 - primer
TGCCTGGCAGTTTATGAAGACGCCTCCGCCCAGCACGGTCAGG
SEQ ID NO: 36 - primer
GCTGATACCGCTCGGAAGACTTAGGAGACGCTTTTTATCGCAACTCTCTACTGTGAA
TTCATTAAAGAGGAGAAAGGTCATATGAAAATATACTCAGCGCTATTGC
SEQ ID NO: 37 - primer
TGCCTGGCAGTTTATGAAGACGCATCATAATCGACTCGTAGATAGC
SEQ ID NO: 38 - primer
GCTGATACCGCTCGGAAGACTTTGATGGAGGTGGCGGATCTGGT
SEQ ID NO: 39 - primer
TGCCTGGCAGTTTATGAAGACGCGCACTTAGCCCAGCACGGTCAGG
SEQ ID NO: 40 - primer
AAGTCTTCCGAGCGGTATCAGC
SEQ ID NO: 41 - primer
GCGTCTTCATAAACTGCCAGGCA

What is claimed is:

1. A chimeric nucleic acid comprising:
(a) a first nucleic acid encoding an ompA protein or a fragment thereof;
(b) a second nucleic acid encoding a radiohapten binding protein.

2. The chimeric nucleic acid of claim 1, wherein the radiohapten binding protein is a 2,2′,2″,2″′-(1,4,7,10-tetraazacyclododecane-1,4,7,10-tetrayl)tetraacetic acid (DOTA)-binding protein.

3. The chimeric nucleic acid of claim 2, wherein the DOTA-binding protein is an anti-DOTA single chain variable fragment (scFV).

4. The chimeric nucleic acid of claim 1, wherein the first nucleic acid sequence comprises SEQ ID NO: 4, or a sequence at least about 70% identical thereto.

5. The chimeric nucleic acid of claim 1, wherein the second nucleic acid comprises SEQ ID NO: 2, or a sequence at least about 70% identical thereto.

6. The chimeric nucleic acid of claim 1, wherein the ompA protein or a fragment thereof is operably linked to the radiohapten binding protein by a linker.

7. The chimeric nucleic acid of claim 6, wherein the linker comprises SEQ ID NO: 5 (GGGGSGGGGSGGGGS).

8. The chimeric nucleic acid of claim 1, comprising the sequence SEQ ID NO: 13, or a sequence at least about 70% identical thereto.

9. A chimeric protein, wherein the chimeric protein is encoded by the chimeric nucleic acid of claim 1.

10. A plasmid or a vector comprising:
(i) the chimeric nucleic acid of claim 1; and
(ii) a heterologous nucleic acid encoding a disulfide bond forming (dsbA) protein.

11. The plasmid or vector of claim 10, wherein the heterologous nucleic acid comprises SEQ ID NO: 15, or a sequence at least about 70% identical thereto.

12. A cell comprising (i) the chimeric nucleic acid of claim 1 or (ii) the plasmid or vector of claim 10.

13. An engineered bacterium comprising a chimeric nucleic acid, wherein the chimeric nucleic acid comprises:
(a) a first nucleic acid encoding an ompA protein or a fragment thereof; and
(b) a second nucleic acid encoding a radiohapten binding protein.

14. The engineered bacterium of claim 13, wherein the engineered bacterium is a Salmonella bacterium or a Salmonella typhimurium bacterium.

15. The engineered bacterium of claim 13, wherein the engineered bacterium comprises the sequence SEQ ID NO: 13, or a sequence at least about 70% identical thereto.

16. The engineered bacterium of claim 13, further comprising a heterologous nucleic acid encoding a dsbA protein.

17. A method of pre-targeted radiotherapy in a subject, comprising administering to the subject the engineered bacterium of claim 13.

18. A method of treating a tumor in a subject in need thereof, comprising administering to the subject an engineered bacterium comprising:
(i) a chimeric nucleic acid, wherein the chimeric nucleic acid comprises:
(a) a first nucleic acid encoding an ompA protein or a fragment thereof; and
(b) a second nucleic acid encoding a radiohapten binding protein; and
(ii) a heterologous nucleic acid encoding a dsbA protein.

19. The method of claim 18, wherein the tumor is a solid tumor.

20. The method of claim 18, wherein the tumor is selected from a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer, a breast cancer, an ovarian cancer, a prostate cancer, a squamous cell carcinoma, a basal cell carcinoma, an adenocarcinoma, a sweat gland carcinoma, a sebaceous gland carcinoma, a papillary carcinoma, a papillary adenocarcinoma, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer, a uterine cancer, a testicular cancer, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma


SEQUENCE LISTING (41 sequences)

SEQ ID NO: 1 - protein, 244 aa, synthetic construct
  REGION 1..244: note = 2D12.5 scFv
QVKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSSGGGGSGGGGSGGGGSQAVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTRLTVLG

SEQ ID NO: 2 - DNA, 735 bp, synthetic construct
  source 1..735: mol_type = other DNA
  misc_feature 1..735: note = 2D12.5 scFv
caggtgaaactgcaggaaagcggcccgggcctggtgcagccgagccagagcctgagcattacctgcaccgtgagcggctttagcctgaccgattatggcgtgcattgggtgcgccagagcccgggcaaaggcctggaatggctgggcgtgatttggagcggcggcggcaccgcgtataccgcggcgtttattagccgcctgaacatttataaagataacagcaaaaaccaggtgttttttgaaatgaacagcctgcaggcgaacgataccgcgatgtattattgcgcgcgccgcggcagctatccgtataactattttgatgtgtggggccagggcaccaccgtgaccgtgagcagcggtggaggtggtagcggtggaggaggtagcggtggaggtggtagccaggcggtggtgacccaggaaagcgcgctgaccaccagcccgggcgaaaccgtgaccctgacctgccgcagcagcaccggcgcggtgaccaccagcaactatgcgaactgggtgcaggaaaaaccggatcatctgtttaccggcctgattggcggcaacaacaaccgcccgccgggcgtgccggcgcgctttagcggcagcctgattggcgataaagcggcgctgaccattgcgggcacccagaccgaagatgaagcgatttatttttgcgcgctgtggtatagcaaccattgggtgtttggcggcggcacccgcctgaccgtgctgggctaa

SEQ ID NO: 3 - protein, 118 aa, synthetic construct
  REGION 1..118: note = ompA
NPYVGFEMGYDWLGRMPYKGDNINGAYKAQGVQLTAKLGYPITDDLDVYTRLGGMVWRADTKSNVPGGPSTKDHDTGVSPVFAGGIEYAITPEIATRLEYQWTNNIGDANTIGTRPDN

SEQ ID NO: 4 - DNA, 354 bp, synthetic construct
  source 1..354: mol_type = other DNA
  misc_feature 1..354: note = ompA
aacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaac

SEQ ID NO: 5 - protein, 14 aa, synthetic construct
  REGION 1..14: note = linker 1
GGGSGGGGSGGGGS

SEQ ID NO: 6 - DNA, 42 bp, synthetic construct
  source 1..42: mol_type = other DNA
  misc_feature 1..42: note = linker 1
ggaggtggtagtggtggaggtggaagtggtggaggaggcagc

SEQ ID NO: 7 - protein, 15 aa, synthetic construct
  REGION 1..15: note = linker 2
GGGGSGGGGRGGGGR

SEQ ID NO: 8 - DNA, 87 bp, synthetic construct
  source 1..87: mol_type = other DNA
  misc_feature 1..87: note = linker 2
ggaggtggcggatctggtggaggtggcagaggtggaggtggcagagactacaaagaccatgacggtgattataaagatcatgacatc

SEQ ID NO: 9 - protein, 22 aa, synthetic construct
  NON_STD 1..22: note = flag tag
DYKDHDGDYKDHDIDYKDDDDK

SEQ ID NO: 10 - DNA, 24 bp, synthetic construct
  source 1..24: mol_type = other DNA
  misc_feature 1..24: note = flag tag
gattacaaggatgacgatgacaag

SEQ ID NO: 11 - protein, 51 aa, synthetic construct
  REGION 1..51: note = full linker
GGGGSGGGGRGGGGRDYKDHDGDYKDHDIDYKDDDDKGGGSGGGGSGGGGS

SEQ ID NO: 12 - DNA, 153 bp, synthetic construct
  source 1..153: mol_type = other DNA
  misc_feature 1..153: note = full linker
ggaggtggcggatctggtggaggtggcagaggtggaggtggcagagactacaaagaccatgacggtgattataaagatcatgacatcgattacaaggatgacgatgacaagggaggtggtagtggtggaggtggaagtggtggaggaggcagc

SEQ ID NO: 13 - DNA, 1329 bp, synthetic construct
  source 1..1329: mol_type = other DNA
  misc_feature 1..1329: note = lpp-ompA-flag-2D12.5
atgaaagctactaaactggtactgggcgcggtaatcctgggttctactctgctggcaggttgctccagcaacgctaaaatcgatcagaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggaggtggcggatctggtggaggtggcagaggtggaggtggcagagactacaaagaccatgacggtgattataaagatcatgacatcgattacaaggatgacgatgacaagggaggtggtagtggtggaggtggaagtggtggaggaggcagccaggtgaaactgcaggaaagcggcccgggcctggtgcagccgagccagagcctgagcattacctgcaccgtgagcggctttagcctgaccgattatggcgtgcattgggtgcgccagagcccgggcaaaggcctggaatggctgggcgtgatttggagcggcggcggcaccgcgtataccgcggcgtttattagccgcctgaacatttataaagataacagcaaaaaccaggtgttttttgaaatgaacagcctgcaggcgaacgataccgcgatgtattattgcgcgcgccgcggcagctatccgtataactattttgatgtgtggggccagggcaccaccgtgaccgtgagcagcggtggaggtggtagcggtggaggaggtagcggtggaggtggtagccaggcggtggtgacccaggaaagcgcgctgaccaccagcccgggcgaaaccgtgaccctgacctgccgcagcagcaccggcgcggtgaccaccagcaactatgcgaactgggtgcaggaaaaaccggatcatctgtttaccggcctgattggcggcaacaacaaccgcccgccgggcgtgccggcgcgctttagcggcagcctgattggcgataaagcggcgctgaccattgcgggcacccagaccgaagatgaagcgatttatttttgcgcgctgtggtatagcaaccattgggtgtttggcggcggcacccgcctgaccgtgctgggctaa

SEQ ID NO: 14 - protein, 442 aa, synthetic construct
  REGION 1..442: note = lpp-ompA-flag-2D12.5
MKATKLVLGAVILGSTLLAGCSSNAKIDQNPYVGFEMGYDWLGRMPYKGDNINGAYKAQGVQLTAKLGYPITDDLDVYTRLGGMVWRADTKSNVPGGPSTKDHDTGVSPVFAGGIEYAITPEIATRLEYQWTNNIGDANTIGTRPDNGGGGSGGGGRGGGGRDYKDHDGDYKDHDIDYKDDDDKGGGSGGGGSGGGGSQVKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSSGGGGSGGGGSGGGGSQAVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTRLTVLG

SEQ ID NO: 15 - DNA, 651 bp, synthetic construct
  source 1..651: mol_type = other DNA
  misc_feature 1..651: note = dsbA
atgaaaaagatttggctggcgctggctggtatggttttagcttttagcgcctcggcagcacagatcagcgacggtaaacagtatatcacgctggataaaccggtcgctggcgaaccccaggtactggagtttttctccttctactgcccacattgttatcagtttgaagaagtgcttcatgtgtctgacaatgtgaagaaaaagctgccggaaggcaccaaaatgaccaagtaccacgttgagttcctggggccgttgggcaaggagctcacccaggcatgggcggtggcgatggcgttgggtgtagaagataaagtcacggtcccgctgtttgaagccgtacagaaaacccagacagtacaatctgccgcggatatccgtaaagtgttcgttgatgcgggcgtcaagggcgaagattacgatgcggcatggaacagcttcgtggtgaaatcactggttgcgcaacaggagaaagccgcggctgacctgcaactgcagggcgttccggcgatgttcgtcaatggcaaataccagattaacccacaaggcatggatacgagcagcatggatgtttttgttcagcagtatgctgatactgtgaaatatttggttgataaaaaagaacaaaaactcatctcagaagaggatctg

SEQ ID NO: 16 - protein, 217 aa, synthetic construct
  REGION 1..217: note = dsbA
MKKIWLALAGMVLAFSASAAQISDGKQYITLDKPVAGEPQVLEFFSFYCPHCYQFEEVLHVSDNVKKKLPEGTKMTKYHVEFLGPLGKELTQAWAVAMALGVEDKVTVPLFEAVQKTQTVQSAADIRKVFVDAGVKGEDYDAAWNSFVVKSLVAQQEKAAADLQLQGVPAMFVNGKYQINPQGMDTSSMDVFVQQYADTVKYLVDKKEQKLISEEDL

SEQ ID NO: 17 - DNA, 1173 bp, synthetic construct
  source 1..1173: mol_type = other DNA
  misc_feature 1..1173: note = asd
atggtgaaggatgcgccacaggatactggcgcgcatacacagcacatctctttgcaggaaaaaaacgctatgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccggcacgctacaggacgcttttgacctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaaaacctttgtgggcggtaactgtaccgttagcctgatgttgatgtcgctgggcggtctgtttgcccataatctcgttgactgggtatccgttgcaacttatcaagcagcttctggtggtggtgcacgtcatatgcgtgagctgttaacccagatgggtcagttgtatggccatgtcgccgatgaactggcgacgccgagctccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag

SEQ ID NO: 18 - DNA, 89 bp, synthetic construct
  source 1..89: mol_type = other DNA
  misc_feature 1..89: note = primer
agctcccggagacggtcgacgctttttatcgcaactctctactgtaaaaaaaaaaggaggaaaaaaaatgaaaaagatttggctggcgc

SEQ ID NO: 19 - DNA, 84 bp, synthetic construct
  source 1..84: mol_type = other DNA
  misc_feature 1..84: note = primer
atccgcttacagacaagctgtttacagatcctcttctgagatgagtttttgttcttttttatcaaccaaatatttcacagtatc

SEQ ID NO: 20 - DNA, 21 bp, synthetic construct
  source 1..21: mol_type = other DNA
  misc_feature 1..21: note = primer
acagcttgtctgtaagcggat

SEQ ID NO: 21 - DNA, 17 bp, synthetic construct
  source 1..17: mol_type = other DNA
  misc_feature 1..17: note = primer
gaccgtctccgggagct

SEQ ID NO: 22 - DNA, 42 bp, synthetic construct
  source 1..42: mol_type = other DNA
  misc_feature 1..42: note = primer
atttcacaccgcatatggtgccgtacgttttcgttccattgg

SEQ ID NO: 23 - DNA, 45 bp, synthetic construct
  source 1..45: mol_type = other DNA
  misc_feature 1..45: note = primer
cagagcagattgtactgagagtatctgcgtttactcctgtattac

SEQ ID NO: 24 - DNA, 22 bp, synthetic construct
  source 1..22: mol_type = other DNA
  misc_feature 1..22: note = primer
actctcagtacaatctgctctg

SEQ ID NO: 25 - DNA, 21 bp, synthetic construct
  source 1..21: mol_type = other DNA
  misc_feature 1..21: note = primer
gcaccatatgcggtgtgaaat

SEQ ID NO: 26 - protein, 29 aa, synthetic construct
  REGION 1..29: note = lpp
MKATKLVLGAVILGSTLLAGCSSNAKIDQ

SEQ ID NO: 27 - DNA, 80 bp, synthetic construct
  source 1..80: mol_type = other DNA
  misc_feature 1..80: note = lpp
atgaaagctactaaactggtactgggcgcggtaatcctgggttctactctgctggcaggttgctccagcaacgctaaaat

SEQ ID NO: 28 - DNA, 5423 bp, synthetic construct
  source 1..5423: mol_type = other DNA
  misc_feature 1..5423: note = full plasmid without dsbA
atgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgaaagctactaaactggtactgggcgcggtaatcctgggttctactctgctggcaggttgctccagcaacgctaaaatcgatcagaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggaggtggcggatctggtggaggtggcagaggtggaggtggcagagactacaaagaccatgacggtgattataaagatcatgacatcgattacaaggatgacgatgacaagggaggtggtagtggtggaggtggaagtggtggaggaggcagccaggtgaaactgcaggaaagcggcccgggcctggtgcagccgagccagagcctgagcattacctgcaccgtgagcggctttagcctgaccgattatggcgtgcattgggtgcgccagagcccgggcaaaggcctggaatggctgggcgtgatttggagcggcggcggcaccgcgtataccgcggcgtttattagccgcctgaacatttataaagataacagcaaaaaccaggtgttttttgaaatgaacagcctgcaggcgaacgataccgcgatgtattattgcgcgcgccgcggcagctatccgtataactattttgatgtgtggggccagggcaccaccgtgaccgtgagcagcggtggaggtggtagcggtggaggaggtagcggtggaggtggtagccaggcggtggtgacccaggaaagcgcgctgaccaccagcccgggcgaaaccgtgaccctgacctgccgcagcagcaccggcgcggtgaccaccagcaactatgcgaactgggtgcaggaaaaaccggatcatctgtttaccggcctgattggcggcaacaacaaccgcccgccgggcgtgccggcgcgctttagcggcagcctgattggcgataaagcggcgctgaccattgcgggcacccagaccgaagatgaagcgatttatttttgcgcgctgtggtatagcaaccattgggtgtttggcggcggcacccgcctgaccgtgctgggctaaatggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataacc

SEQ ID NO: 29 - DNA, 7644 bp, synthetic construct
  source 1..7644: mol_type = other DNA
  misc_feature 1..7644: note = full plasmid with dsbA
acagtagagagttgcgataaaaagcgtcgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgaaagctactaaactggtactgggcgcggtaatcctgggttctactctgctggcaggttgctccagcaacgctaaaatcgatcagaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggaggtggcggatctggtggaggtggcagaggtggaggtggcagagactacaaagaccatgacggtgattataaagatcatgacatcgattacaaggatgacgatgacaagggaggtggtagtggtggaggtggaagtggtggaggaggcagccaggtgaaactgcaggaaagcggcccgggcctggtgcagccgagccagagcctgagcattacctgcaccgtgagcggctttagcctgaccgattatggcgtgcattgggtgcgccagagcccgggcaaaggcctggaatggctgggcgtgatttggagcggcggcggcaccgcgtataccgcggcgtttattagccgcctgaacatttataaagataacagcaaaaaccaggtgttttttgaaatgaacagcctgcaggcgaacgataccgcgatgtattattgcgcgcgccgcggcagctatccgtataactattttgatgtgtggggccagggcaccaccgtgaccgtgagcagcggtggaggtggtagcggtggaggaggtagcggtggaggtggtagccaggcggtggtgacccaggaaagcgcgctgaccaccagcccgggcgaaaccgtgaccctgacctgccgcagcagcaccggcgcggtgaccaccagcaactatgcgaactgggtgcaggaaaaaccggatcatctgtttaccggcctgattggcggcaacaacaaccgcccgccgggcgtgccggcgcgctttagcggcagcctgattggcgataaagcggcgctgaccattgcgggcacccagaccgaagatgaagcgatttatttttgcgcgctgtggtatagcaaccattgggtgtttggcggcggcacccgcctgaccgtgctgggctaaatggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgccgtacgttttcgttccattggccctcaaacccctaattaggatcaataaaacagcgacggaaatgattcccttcctaacgcaaattccctgataatcgccactggactttctgcttgcgcggtaaggcaggataagtcgcattactgatggcttcgctatcattgattaatttcacttgcgactttggctgctttttgtatggtgaaggatgcgccacaggatactggcgcgcatacacagcacatctctttgcaggaaaaaaacgctatgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccggcacgctacaggacgcttttgacctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaaaacctttgtgggcggtaactgtaccgttagcctgatgttgatgtcgctgggcggtctgtttgcccataatctcgttgactgggtatccgttgcaacttatcaagcagcttctggtggtggtgcacgtcatatgcgtgagctgttaacccagatgggtcagttgtatggccatgtcgccgatgaactggcgacgccgagctccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtggctattgcagcgcttatcgggcctgcgtgtggttctgtaggccggataaggcgcgtcagcgccgccatccggcggggaaatttgtgttaaaccaggggtgcatcgtcaccctttttttgcgtaatacaggagtaaacgcagatactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtttacagatcctcttctgagatgagtttttgttcttttttatcaaccaaatatttcacagtatcagcatactgctgaacaaaaacatccatgctgctcgtatccatgccttgtgggttaatctggtatttgccattgacgaacatcgccggaacgccctgcagttgcaggtcagccgcggctttctcctgttgcgcaaccagtgatttcaccacgaagctgttccatgccgcatcgtaatcttcgcccttgacgcccgcatcaacgaacactttacggatatccgcggcagattgtactgtctgggttttctgtacggcttcaaacagcgggaccgtgactttatcttctacacccaacgccatcgccaccgcccatgcctgggtgagctccttgcccaacggccccaggaactcaacgtggtacttggtcattttggtgccttccggcagctttttcttcacattgtcagacacatgaagcacttcttcaaactgataacaatgtgggcagtagaaggagaaaaactccagtacctggggttcgccagcgaccggtttatccagcgtgatatactgtttaccgtcgctgatctgtgctgccgaggcgctaaaagctaaaaccataccagccagcgccagccaaatctttttcattttttttcctcctttttttttt

SEQ ID NO: 30 - DNA, 47 bp, synthetic construct
  source 1..47: mol_type = other DNA
  misc_feature 1..47: note = primer
gctgataccgctcggaagacttcagctgggttgacgttgatagtctg

SEQ ID NO: 31 - DNA, 53 bp, synthetic construct
  source 1..53: mol_type = other DNA
  misc_feature 1..53: note = primer
tgcctggcagtttatgaagacgcgcacttaatcgttatcaatagcattgtggc

SEQ ID NO: 32 - DNA, 39 bp, synthetic construct
  source 1..39: mol_type = other DNA
  misc_feature 1..39: note = primer
gctgataccgctcggaagacttggaggtggcggatctgg

SEQ ID NO: 33 - DNA, 40 bp, synthetic construct
  source 1..40: mol_type = other DNA
  misc_feature 1..40: note = primer
tgcctggcagtttatgaagacgcgctgcctcctccaccac

SEQ ID NO: 34 - DNA, 100 bp, synthetic construct
  source 1..100: mol_type = other DNA
  misc_feature 1..100: note = primer
gctgataccgctcggaagacttaggatttacactttatgcttccggctcgtataatgaaaaaaaaaggaggaaaaaaatgcaggtgaaactgcaggaaag

SEQ ID NO: 35 - DNA, 43 bp, synthetic construct
  source 1..43: mol_type = other DNA
  misc_feature 1..43: note = primer
tgcctggcagtttatgaagacgcctccgcccagcacggtcagg

SEQ ID NO: 36 - DNA, 106 bp, synthetic construct
  source 1..106: mol_type = other DNA
  misc_feature 1..106: note = primer
gctgataccgctcggaagacttaggagacgctttttatcgcaactctctactgtgaattcattaaagaggagaaaggtcatatgaaaatatactcagcgctattgc

SEQ ID NO: 37 - DNA, 46 bp, synthetic construct
  source 1..46: mol_type = other DNA
  misc_feature 1..46: note = primer
tgcctggcagtttatgaagacgcatcataatcgactcgtagatagc

SEQ ID NO: 38 - DNA, 44 bp, synthetic construct
  source 1..44: mol_type = other DNA
  misc_feature 1..44: note = primer
gctgataccgctcggaagactttgatggaggtggcggatctggt

SEQ ID NO: 39 - DNA, 46 bp, synthetic construct
  source 1..46: mol_type = other DNA
  misc_feature 1..46: note = primer
tgcctggcagtttatgaagacgcgcacttagcccagcacggtcagg

SEQ ID NO: 40 - DNA, 22 bp, synthetic construct
  source 1..22: mol_type = other DNA
  misc_feature 1..22: note = primer
aagtcttccgagcggtatcagc

SEQ ID NO: 41 - DNA, 23 bp, synthetic construct
  source 1..23: mol_type = other DNA
  misc_feature 1..23: note = primer
gcgtcttcataaactgccaggca